SEQUENCE LISTING (2 sequences)

SEQ ID NO: 1 - DNA, 4206 bp, Homo sapiens
  source 1..4206: mol_type = genomic DNA
ataactttgtagcgagtcgaaaactgaggctccggccgcagagaactcagcctcattcctgctttaaaatctctcggccacctttgatgaggggactgggcagttctagacagtcccgaagttctcaaggcacaggtctcttcctggtttgactgtccttaccccggggaggcagtgcagccagctgcaagccccacagtgaagaacatctgagctcaaatccagataagtgacataagtgacctgctttgtaaagccatagagatggcctgtccttggaaatttctgttcaagaccaaattccaccagtatgcaatgaatggggaaaaagacatcaacaacaatgtggagaaagccccctgtgccacctccagtccagtgacacaggatgaccttcagtatcacaacctcagcaagcagcagaatgagtccccgcagcccctcgtggagacgggaaagaagtctccagaatctctggtcaagctggatgcaaccccattgtcctccccacggcatgtgaggatcaaaaactggggcagcgggatgactttccaagacacacttcaccataaggccaaagggattttaacttgcaggtccaaatcttgcctggggtccattatgactcccaaaagtttgaccagaggacccagggacaagcctacccctccagatgagcttctacctcaagctatcgaatttgtcaaccaatattacggctccttcaaagaggcaaaaatagaggaacatctggccagggtggaagcggtaacaaaggagatagaaacaacaggaacctaccaactgacgggagatgagctcatcttcgccaccaagcaggcctggcgcaatgccccacgctgcattgggaggatccagtggtccaacctgcaggtcttcgatgcccgcagctgttccactgcccgggaaatgtttgaacacatctgcagacacgtgcgttactccaccaacaatggcaacatcaggtcggccatcaccgtgttcccccagcggagtgatggcaagcacgacttccgggtgtggaatgctcagctcatccgctatgctggctaccagatgccagatggcagcatcagaggggaccctgccaacgtggaattcactcagctgtgcatcgacctgggctggaagcccaagtacggccgcttcgatgtggtccccctggtcctgcaggccaatggccgtgaccctgagctcttcgaaatcccacctgaccttgtgcttgaggtggccatggaacatcccaaatacgagtggtttcgggaactggagctaaagtggtacgccctgcctgcagtggccaacatgctgcttgaggtgggcggcctggagttcccagggtgccccttcaatggctggtacatgggcacagagatcggagtccgggacttctgtgacgtccagcgctacaacatcctggaggaagtgggcaggagaatgggcctggaaacgcacaagctggcctcgctctggaaagaccaggctgtcgttgagatcaacattgctgtgctccatagtttccagaagcagaatgtgaccatcatggaccaccactcggctgcagaatccttcatgaagtacatgcagaatgaataccggtcccgtgggggctgcccggcagactggatttggctggtccctcccatgtctgggagcatcacccccgtgtttcaccaggagatgctgaactacgtcctgtcccctttctactactatcaggtagaggcctggaaaacccatgtctggcaggacgagaagcggagacccaagagaagagagattccattgaaagtcttggtcaaagctgtgctctttgcctgtatgctgatgcgcaagacaatggcgtcccgagtcagagtcaccatcctctttgcgacagagacaggaaaatcagaggcgctggcctgggacctgggggccttattcagctgtgccttcaaccccaaggttgtctgcatggataagtacaggctgagctgcctggaggaggaacggctgctgttggtggtgaccagtacgtttggcaatggagactgccctggcaatggagagaaactgaagaaatcgctcttcatgctgaaagagctcaacaacaaattcaggtacgctgtgtttggcctcggctccagcatgtaccctcggttctgcgcctttgctcatgacattgatcagaagctgtcccacctgggggcctctcagctcaccccgatgggagaaggggatgagctcagtgggcaggaggacgccttccgcagctgggccgtgcaaaccttcaaggcagcctgtgagacgtttgatgtccgaggcaaacagcacattcagatccccaagctctacacctccaatgtgacctgggacccgcaccactacaggctcgtgcaggactcacagcctttggacctcagcaaagccctcagcagcatgcatgccaagaacgtgttcaccatgaggctcaaatctcggcagaatctacaaagtccgacatccagccgtgccaccatcctggtggaactctcctgtgaggatggccaaggcctgaactacctgccgggggagcaccttggggtttgcccaggcaaccagccggccctggtccaaggtatcctggagcgagtggtggatggccccacaccccaccagacagtgcgcctggaggccctggatgagagtggcagctactgggtcagtgacaagaggctgcccccctgctcactcagccaggccctcacctacttcctggacatcaccacacccccaacccagctgctgctccaaaagctggcccaggtggccacagaagagcctgagagacagaggctggaggccctgtgccagccctcagagtacagcaagtggaagttcaccaacagccccacattcctggaggtgctagaggagttcccgtccctgcgggtgtctgctggcttcctgctttcccagctccccattctgaagcccaggttctactccatcagctcctcccgggatcacacgcccacagagatccacctgactgtggccgtggtcacctaccacacccgagatggccagggtcccctgcaccacggcgtctgcagcacatggctcaacagcctgaagccccaagacccagtgccctgctttgtgcggaatgccagcggcttccacctccccgaggatccctcccatccttgcatcctcatcgggcctggcacaggcatcgcgcccttccgcagtttctggcagcaacggctccatgactcccagcacaagggagtgcggggaggccgcatgaccttggtgtttgggtgccgccgcccagatgaggaccacatctaccaggaggagatgctggagatggcccagaagggggtgctgcatgcggtgcacacagcctattcccgcctgcctggcaagcccaaggtctatgttcaggacatcctgcggcagcagctggccagcgaggtgctccgtgtgctccacaaggagccaggccacctctatgtttgcggggatgtgcgcatggcccgggacgtggcccacaccctgaagcagctggtggctgccaagctgaaattgaatgaggagcaggtcgaggactatttctttcagctcaagagccagaagcgctatcacgaagatatctttggtgctgtatttccttacgaggcgaagaaggacagggtggcggtgcagcccagcagcctggagatgtcagcgctctgagggcctacaggaggggttaaagctgccggcacagaacttaaggatggagccagctctgcattatctgaggtcacagggcctggggagatggaggaaagtgatatcccccagcctcaagtcttatttcctcaacgttgctccccatcaagccctttacttgacctcctaacaagtagcaccctggattgatcggagcctcctctctcaaactggggcctccctggtcccttggagacaaaatcttaaatgccaggcctggcaagtgggtgaaagatggaacttgctgctgagtgcaccacttcaagtgaccaccaggaggtgctatcgcaccactgtgtatttaactgccttgtgtacagttatttatgcctctgtatttaaaaaactaacacccagtctgttccccatggccacttgggtcttccctgtatgattccttgatggagatatttacatgaattgcattttactttaatcaca

SEQ ID NO: 2 - protein, 1153 aa, Homo sapiens
MACPWKFLFKTKFHQYAMNGEKDINNNVEKAPCATSSPVTQDDLQYHNLSKQQNESPQPLVETGKKSPESLVKLDATPLSSPRHVRIKNWGSGMTFQDTLHHKAKGILTCRSKSCLGSIMTPKSLTRGPRDKPTPPDELLPQAIEFVNQYYGSFKEAKIEEHLARVEAVTKEIETTGTYQLTGDELIFATKQAWRNAPRCIGRIQWSNLQVFDARSCSTAREMFEHICRHVRYSTNNGNIRSAITVFPQRSDGKHDFRVWNAQLIRYAGYQMPDGSIRGDPANVEFTQLCIDLGWKPKYGRFDVVPLVLQANGRDPELFEIPPDLVLEVAMEHPKYEWFRELELKWYALPAVANMLLEVGGLEFPGCPFNGWYMGTEIGVRDFCDVQRYNILEEVGRRMGLETHKLASLWKDQAVVEINIAVLHSFQKQNVTIMDHHSAAESFMKYMQNEYRSRGGCPADWIWLVPPMSGSITPVFHQEMLNYVLSPFYYYQVEAWKTHVWQDEKRRPKRREIPLKVLVKAVLFACMLMRKTMASRVRVTILFATETGKSEALAWDLGALFSCAFNPKVVCMDKYRLSCLEEERLLLVVTSTFGNGDCPGNGEKLKKSLFMLKELNNKFRYAVFGLGSSMYPRFCAFAHDIDQKLSHLGASQLTPMGEGDELSGQEDAFRSWAVQTFKAACETFDVRGKQHIQIPKLYTSNVTWDPHHYRLVQDSQPLDLSKALSSMHAKNVFTMRLKSRQNLQSPTSSRATILVELSCEDGQGLNYLPGEHLGVCPGNQPALVQGILERVVDGPTPHQTVRLEALDESGSYWVSDKRLPPCSLSQALTYFLDITTPPTQLLLQKLAQVATEEPERQRLEALCQPSEYSKWKFTNSPTFLEVLEEFPSLRVSAGFLLSQLPILKPRFYSISSSRDHTPTEIHLTVAVVTYHTRDGQGPLHHGVCSTWLNSLKPQDPVPCFVRNASGFHLPEDPSHPCILIGPGTGIAPFRSFWQQRLHDSQHKGVRGGRMTLVFGCRRPDEDHIYQEEMLEMAQKGVLHAVHTAYSRLPGKPKVYVQDILRQQLASEVLRVLHKEPGHLYVCGDVRMARDVAHTLKQLVAAKLKLNEEQVEDYFFQLKSQKRYHEDIFGAVFPYEAKKDRVAVQPSSLEMSAL

(19) United States
(12) Patent Application Publication
(10) Pub. No.: US 2023/0220479 A1
(43) Pub. Date: Jul. 13, 2023

(54) BIOMARKERS AND CLASSIFIER OF PSORIASIS AND METHODS OF TREATMENT

(71) Applicant: Icahn School of Medicine at Mount Sinai, New York, NY (US)

(72) Inventors: Ana Brandusa Pavel, New York, NY (US); Emma Guttman-Yassky, New York, NY (US)

(21) Appl. No.: 18/155,702

(22) Filed: Jan. 17, 2023

Related U.S. Application Data
(63) Continuation-in-part of application No. PCT/US2021/041796, filed on Jul. 15, 2021.
(60) Provisional application No. 63/053,361, filed on Jul. 17, 2020.

(51) Int. Cl. C12Q 1/6883; G01N 33/68

(52) U.S. Cl. CPC C12Q 1/6883; G01N 33/6881; G01N 2800/202; G01N 2800/205; G01N 2333/90254; C12Q 2600/158

(57) ABSTRACT

The present invention features biomarkers for diagnosis and treatment of dermatological diseases, e.g., psoriasis and atopic dermatitis. The disclosure provides a minimally invasive method of determining levels of biomarkers (e.g., NOS2/iNOS) in surface skin samples.

### CROSS-REFERENCE TO RELATED APPLICATIONS

This application is a continuation-in-part of International Application No. PCT/US2021/041796, filed Jul. 15, 2021 (published as WO 2022/015960 on Jan. 20, 2022), which claims the benefit of U.S. Provisional Application No. 63/053,361, filed Jul. 17, 2020; each of which are incorporated herein by reference in its entirety.

### SEQUENCE LISTING

This application contains a Sequence Listing that has been submitted electronically as an XML file named “27527-0178001_SL_ST26.XML.” The XML file, created on Jan. 12, 2023, is 8,117 bytes in size. The material in the XML file is hereby incorporated by reference in its entirety.

### TECHNICAL FIELD

This disclosure relates generally to biomarkers of dermatologic disorders, e.g., psoriasis and atopic dermatitis (AD), and to methods of diagnosing and treating dermatologic disorders, e.g., psoriasis and AD, based on NOS2/iNOS levels in a subject.

### BACKGROUND

Psoriasis is a chronic, multisystem inflammatory disease with predominantly skin and joint involvement. It is also associated with a number of comorbidities, including psoriatic arthritis, cardiovascular disease, diabetes, malignancy, depression, and anxiety, that requires timely therapy to improve long-term outcomes. Psoriasis affects at least 100 million individuals worldwide. The diagnosis of psoriasis is primarily clinical and a skin biopsy may be required. Apart from clinical examination of the skin, scalp and nails, and skin biopsies, there are no special blood tests or tools to diagnose psoriasis. Despite its considerable effect on quality of life, psoriasis is underdiagnosed and undertreated. Psoriasis may also be misdiagnosed as atopic dermatitis (AD) or eczema.

Skin biopsies may be used to better diagnose psoriasis based on the molecular profile of the biopsied skin. However, skin biopsies are known to cause scarring, pain, and carry a risk of infection, limiting their utility in large clinical trials and longitudinal studies, especially in children. Thus, a less invasive means to accurately detect disease profiles in skin is needed. Further, there is a need for biomarkers for timely and accurate diagnosis of psoriasis.

### SUMMARY

Inducible nitric oxide synthase (iNOS or NOS2) is an enzyme that produces nitrous oxide upon stimulation by pro-inflammatory cytokines. NOS2/iNOS is an innate immunity marker associated with hypergranulosis. This disclosure is based, at least in part, on the finding that NOS2/iNOS levels, as measured in a minimally invasive, non-scarring approach utilizing skin sampling by tape-stripping serves as an effective biomarker for psoriasis and that NOS2/iNOS levels may be used to discriminate psoriasis from other skin conditions, such as atopic dermatitis (AD). Quantification of NOS2/iNOS in tape-strips can potentially help diagnose psoriasis, differentiate psoriasis from other conditions such as AD, predict responsiveness to treatment, and track treatment response.

In one aspect, the disclosure features a method of diagnosing psoriasis in a human subject in need thereof, comprising:

(a) obtaining a surface skin sample from the subject exhibiting a skin lesion;

(b) determining the level of NOS2/iNOS biomarker in the sample,

(c) optionally determining the levels of one or more additional biomarkers selected from the group consisting of IL-17C, IL-1β, IL-6, CXCL8/IL-8, TNFa, IFNγ, CXCL9, IL-17A, IL-17F, IL-23p19, IL-12/IL-23p40, IL-36A, IL-36G, DEFB4B, CCL20, CXCL1, CAMP/LL37, PI3, STAT3, S100A9, and S100A12 in the sample;

(d) comparing the level of the NOS2/iNOS biomarker in the sample to a reference level of NOS2/iNOS biomarker in a control;

(e) optionally comparing the level of the one or more additional biomarkers in step (c) in the sample to reference levels of the corresponding one or more additional biomarkers of step (c) in the control;

wherein the subject has psoriasis if the levels of NOS2/iNOS biomarker and optionally the levels of the one or more additional biomarkers of step (c) in the sample is higher than the reference levels of the corresponding biomarkers in the control.

In another aspect, the disclosure features a method of discriminating between atopic dermatitis and psoriasis in a human subject exhibiting an indeterminate lesion, the method comprising:

(a) obtaining a surface skin sample from the subject exhibiting the skin lesion;

(b) measuring the level of NOS2/iNOS biomarker in the sample;

(c) optionally measuring the level of one or more biomarkers selected from a group consisting of IL-17C, IL-1B, IL-6, CXCL8/IL-8, DEFB4B, CXCL9, SERPINB3, CCL20, S100A9, IL17A, PI3, CXCL10, and IL36A in the sample;

(d) comparing the level of the NOS2/iNOS biomarker to a reference level of NOS2/iNOS biomarker in a control sample;

(e) determining that the subject has psoriasis if the NOS2/iNOS level, and optionally the levels of the one or more additional biomarkers of step (c) in the sample, are higher than the reference levels of NOS2/iNOS biomarker and the one or more additional biomarkers in the control;

(f) determining that the subject has atopic dermatitis if the NOS2/iNOS level, and optionally the levels of the one or more additional biomarkers of step (c) in the sample are similar to or lower than the reference levels in the control.

In a third aspect the disclosure provides a method of treating psoriasis, comprising identifying a human subject having psoriasis who is likely to be responsive to psoriasis therapy, further comprising:

(a) obtaining a surface skin sample from the subject exhibiting a skin lesion;

(b) determining the level of NOS2/iNOS biomarker in the sample;

(c) optionally determining the levels of one or more additional biomarkers selected from the group consisting of IL-17C, IL-1β, IL-6, CXCL8/IL-8, TNFα, IFNγ, CXCL9, IL-17A, IL-17F, IL-23p19, IL-12/IL-23p40, IL-36A, IL-36G, DEFB4B, CCL20, CXCL1, CAMP/LL37, PI3, STAT3, S100A9, and S100A12 in the sample;

(d) comparing the level of the NOS2/iNOS biomarker in the sample to a reference level of NOS2/iNOS biomarker in a control;

(e) optionally comparing the level of the one or more additional biomarkers in step (c) in the sample to reference levels of the corresponding one or more additional biomarkers of step (c) in the control;

(f) determining that the subject has psoriasis if the levels of NOS2/iNOS biomarker and optionally the one or more biomarkers of step (c) is higher than the reference level of NOS2/iNOS biomarkers and optionally the corresponding levels of the one or more additional biomarkers of step (e) of in the control; and administering a therapeutically effective amount of a psoriasis therapy to the subject identified as likely to be responsive to the psoriasis therapy.

In another aspect, the disclosure provides a method of treating psoriasis, comprising discriminating between atopic dermatitis and psoriasis in a human subject exhibiting an indeterminate lesion, further comprising:

(a) obtaining a surface skin sample from the subject exhibiting a skin lesion by tape-strip applied to a selected skin surface, wherein application of the tape-strip to the selected skin surface transfers the surface skin sample to the tape-strip;

(b) determining the level of NOS2/iNOS biomarker in the surface skin sample;

(c) comparing the level of the NOS2/iNOS biomarker in the surface skin sample to a reference level of NOS2/iNOS biomarker in a control sample;

(d) determining that the subject has psoriasis if the levels of NOS2/iNOS biomarker is higher than the reference level of NOS2/iNOS biomarkers in the control sample;

(e) determining that the subject has atopic dermatitis if the NOS2/iNOS level in the surface skin sample are similar to or lower than the reference levels in the control sample; and

(f) administering a therapeutically effective amount of a psoriasis therapy to the human subject identified as having psoriasis.

In some embodiments, the subject does not have psoriasis if the NOS2/iNOS biomarker level in the surface skin sample is zero or negligible. In some embodiments, the subject has psoriasis if the NOS2/iNOS biomarker level in the surface skin sample is not zero or negligible. In some embodiments, more than one tape-strip is obtained from the same skin surface. In some embodiments, at least 3 tape-strips are obtained from the same skin surface. In some embodiments, the selected skin surface is non-lesional skin. In some embodiments, the selected skin surface is lesional skin. In some embodiments, the levels of the NOS2/iNOS biomarker are measured by determining the mRNA levels of the NOS2/iNOS biomarker. In some embodiments, the levels of the NOS2/iNOS biomarker are measured by determining the cDNA levels of the NOS2/iNOS biomarker. In some embodiments, the levels of the NOS2/iNOS biomarker are measured by determining the protein levels of the NOS2/iNOS biomarker. In some embodiments, the psoriasis therapy is at least one therapy selected from a group consisting of topical treatment, phototherapy, oral medication, and a biological drug.

In some embodiments, the topical treatment is one or more agents selected from a cream, lotion, spray, moisturizer, bath salt, immunomodulator, coal tar, anthralin, corticosteroid, and vitamin D. In some embodiments, the cream is calcipotriene cream. In some embodiments, the moisturizer contains one or more of salicylic acid, lactic acid, petroleum jelly, and paraffin. In some embodiments, the immunomodulator is tacrolimis or pimecrolimus. In some embodiments, the biological drug is selected from abatacept, adalimumab, certolizumab, brodalumab, dupilumab, etancercept, golimumab, guselkumab, infliximab, ixekizumab, risankizumab, secukinumab, tildrakizumab and ustekinumab. In some embodiments, the oral medication is selected from methotrexate, acitretin, cyclosporine, and apremilast.

In another aspect, the disclosure provides a method of treating atopic dermatitis, comprising discriminating between atopic dermatitis and psoriasis in a human subject exhibiting an indeterminate lesion, further comprising:
(a) obtaining a surface skin sample from the subject exhibiting a skin lesion by tape-strip applied to a selected skin surface, wherein application of the tape-strip to the selected skin surface transfers the surface skin sample to the tape-strip;
(b) determining the level of NOS2/iNOS biomarker in the surface skin sample;
(c) comparing the level of the NOS2/iNOS biomarker in the surface skin sample to a reference level of NOS2/iNOS biomarker in a control sample;
(d) determining that the subject has psoriasis if the levels of NOS2/iNOS biomarker is higher than the reference level of NOS2/iNOS biomarkers in the control sample;
(e) determining that the subject has atopic dermatitis if the NOS2/iNOS level in the surface skin sample are similar to or lower than the reference levels in the control sample; and
(f) administering a therapeutically effective amount of an atopic dermatitis therapy to the human subject identified as having atopic dermatitis.

In some embodiments, the levels of the NOS2/iNOS biomarker are measured by determining the mRNA levels of the NOS2/iNOS biomarker. In some embodiments, the levels of the NOS2/iNOS biomarker are measured by determining the cDNA levels of the NOS2/iNOS biomarker. In some embodiments, the levels of the NOS2/iNOS biomarker are measured by determining the protein levels of the NOS2/iNOS biomarker. In some embodiments, the atopic dermatitis therapy is at least one therapy selected from a group consisting of topical treatment, phototherapy, oral medication, and a biological drug. In some embodiments, the biological drug is selected from dupilumab, dupixent, traloknumab, upadicitinib, abrocotinib, and lebrikizumab. In some embodiments, the topical treatment is ruxolitinib.

In yet another aspect the disclosure provides a method of predicting whether a human subject with psoriasis will be responsive to a psoriasis treatment, comprising:

obtaining one or more surface skin samples from the subject before, during, or after treatment,

measuring the level of NOS2/iNOS biomarker in the samples;

comparing the level of the NOS2/iNOS biomarker in the samples with each other;

wherein a decreased level of NOS2/iNOS biomarker in the one or more samples taken during or after treatment compared to that in the one or more samples taken before treatment indicates the likelihood of an effective response to the psoriasis treatment in the subject.

In another aspect the disclosure provides a method of monitoring subject response to a psoriasis treatment in a human psoriasis subject in need thereof, comprising:

obtaining one or more surface skin samples from the subject before, during, or after treatment,

measuring the level of NOS2/iNOS biomarker in the samples;

comparing the level of the NOS2/iNOS biomarker in the samples with each other;

wherein a decreased level of NOS2/iNOS biomarker in the one or more samples taken during or after treatment compared to that in the one or more samples taken before treatment indicates an effective response to the psoriasis treatment.

In some embodiments of the methods of treatment, prediction of treatment, or monitoring subject response to treatment, the psoriasis treatment or therapy is at least one therapy selected from a group consisting of topical treatment, phototherapy, oral medication, and a biological drug. The topical treatment may be one or more agents including but not limited to a cream, lotion, spray, moisturizer, bath salt, immunomodulator, coal tar, anthralin, corticosteroid, and vitamin D. In some embodiments, the cream is calcipotriene cream. In some embodiments, the moisturizer contains one or more of salicylic acid, lactic acid, petroleum jelly, and paraffin. In some embodiments, the immunomodulator is tacrolimis or pimecrolimus. In some embodiments, the biological drug may be abatacept, adalimumab, certolizumab, brodalumab, dupilumab, etancercept, golimumab, guselkumab, infliximab, ixekizumab, risankizumab, secukinumab, tildrakizumab or ustekinumab. In some embodiments, the oral medication may be methotrexate, acitretin, cyclosporine, and apremilast.

In some embodiments of any of the aspects, the subject does not have psoriasis if the NOS2/iNOS biomarker level in the sample is zero or negligible. In some embodiments, the subject has psoriasis if the NOS2/iNOS biomarker level in the sample is not zero or negligible.

In some embodiments of any of the aspects, the surface skin sample is obtained by tape-strip applied to a selected skin surface, wherein application of the tape-strip to the selected skin surface transfers the sample to the tape-strip. In some embodiments, more than one tape-strip is obtained from the same skin surface. In some embodiments, at least 3 tape-strips are obtained from the same skin surface. In some embodiments, the selected surface skin surface is non-lesional skin. In other embodiments, the selected surface skin surface is lesional skin.

In some embodiments of any of the aspects, the levels of one or more of the biomarkers are measured by determining the mRNA levels of the biomarkers. In other embodiments, the levels of one or more of the biomarkers are measured by determining the cDNA levels of the biomarkers. In some other embodiments, the levels of one or more of the biomarkers are measured by determining the protein levels of the biomarkers.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A-1F shows the relative mRNA expression (expression fold changes) of innate immunity markers (NOS2/iNOS, IL-17C, IL-1β, IL-6 CXCL8/IL-8, and TNFα) detected in lesional (LS) and non-lesional (NL) tape stripped skin of subjects with atopic dermatitis (AD) and psoriasis (PSO); as well as healthy tape-stripped skin as measured by quantitative real-time PCR. *symbols: significance of comparison to normal (N); @symbols: significance of comparison between lesional (LS) and non-lesional (NL) tape stripped skin; #symbols: significance between PSO LS and AD LS groups; $ symbol: significance between PSO NL vs AD NL groups. 3 identical symbols P<0.001, 2 identical symbols P<0.01, single symbol P<0.05, +P<0.1; AD: Acute Dermatitis; PSO: psoriasis.

FIG. 2 shows the area under the receiver operating characteristic curve (ROC AUC) for NOS2/iNOS molecular classifier in tape strips that accurately discriminates between AD and psoriasis using RNA Sequencing (AUC=0.97) and real-time PCR (AUC=1) platforms. RNA-seq: RNA-sequencing; RT-PCR: real-time polymerase chain reaction; AD: atopic dermatitis.

FIG. 3 shows a summary heatmap of gene expressions of predominately Th2 markers (upper dashed line box surrounding CCR10 through IL2) and predominantly Th1/Th17 biomarkers (lower dashed line box surrounding CDSN through IL3) measured by qRT-PCR. The table shows fold changes in non-lesional versus normal (NL vs N), lesional versus normal (LS vs N), lesional vs lesional (LS vs LS), and non-lesional versus non-lesional (NL vs NL) tape-stripped skin for acute dermatitis (AD) and psoriasis (PSO). Non-dotted shades: up-regulation; Dotted shades: down-regulation; ***P<0.001, **P<0.01, *P<0.05, +P<0.1.

FIG. 4A-4B shows the relative mRNA expression (expression fold changes) of Th1 markers (IFNγ and CXCL9) detected in lesional (LS) and non-lesional (NL) tape stripped skin of subjects with atopic dermatitis (AD) and psoriasis (PSO); as well as healthy tape-stripped skin as measured by quantitative real-time PCR. * symbols: significance of comparison to normal (N); @symbols: significance of comparison between lesional (LS) and non-lesional (NL) tape stripped skin; #symbols: significance between PSO LS and AD LS groups; $ symbol: significance between PSO NL vs AD NL groups. 3 identical symbols P<0.001, 2 identical symbols P<0.01, single symbol P<0.05, +P<0.1; AD: Acute Dermatitis; PSO: psoriasis.

FIG. 5A-5F shows the relative mRNA expression (expression fold changes) of Th17 markers (IL-17A, IL-17F, IL-23p19, IL-12/IL-23p40, IL-36A, IL-36G) detected in lesional (LS) and non-lesional (NL) tape stripped skin of subjects with atopic dermatitis (AD) and psoriasis (PSO); as well as healthy tape-stripped skin as measured by quantitative real-time PCR. *symbols: significance of comparison to normal (N); @symbols: significance of comparison between lesional (LS) and non-lesional (NL) tape stripped skin; #symbols: significance between PSO LS and AD LS groups; $ symbol: significance between PSO NL vs AD NL groups. 3 identical symbols P<0.001, 2 identical symbols P<0.01, single symbol P<0.05, +P<0.1; AD: Acute Dermatitis; PSO: psoriasis.

FIG. 6A-61I shows the relative mRNA expression (expression fold changes) of Th17 markers (DEFB4B, CCL20, CXCL1, CAMP/LL37, PI3, STAT3, S100A9, and S100A12) detected in lesional (LS) and non-lesional (NL) tape stripped skin of subjects with atopic dermatitis (AD) and psoriasis (PSO); as well as healthy tape-stripped skin as measured by quantitative real-time PCR. *symbols: significance of comparison to normal (N); @ symbols: significance of comparison between lesional (LS) and non-lesional (NL) tape stripped skin; #symbols: significance between PSO LS and AD LS groups . . . 3 identical symbols P<0.001, 2 identical symbols P<0.01, single symbol P<0.05, +P<0.1; AD: Acute Dermatitis; PSO: psoriasis.

FIG. 7A-7B shows the relative mRNA expression (expression fold changes) of miscellaneous biomarkers (SERPINB3 and CXCL10) detected in lesional (LS) and non-lesional (NL) tape stripped skin of subjects with atopic dermatitis (AD) and psoriasis (PSO); as well as healthy tape-stripped skin as measured by quantitative real-time PCR. * symbols: significance of comparison to normal (N); @symbols: significance of comparison between lesional (LS) and non-lesional (NL) tape stripped skin; #symbols: significance between PSO LS and AD LS groups. 3 identical symbols P<0.001, 2 identical symbols P<0.01, single P<0.05, +P<0.1; AD: Acute Dermatitis; PSO: psoriasis.

FIG. 8A-8D shows scatter plots of tape-strips versus biopsies. Scatter plots depict RNA-seq log 2 fold-changes (log FCH) for lesional skin versus controls (LS vs N) for (A) atopic dermatitis (AD) and (B) psoriasis (PSO), and non-lesional skin versus controls (NL vs N) for (C) atopic dermatitis (AD) and (D) psoriasis (PSO), between tape-strips from this study and biopsies from a previously published similarly severe cohort. The size of the circle represents the absolute difference (in log 2 fold-change) for each diseased group versus controls.

Like reference symbols in the various drawings indicate like elements.

### DETAILED DESCRIPTION

This disclosure is based, in part, on the surprising finding that NOS2/iNOS can serve as a highly effective biomarker for psoriasis as measured in tape-stripped skin sample.

Unless otherwise defined herein, scientific and technical terms used in this application shall have the meanings that are commonly understood by those of ordinary skill in the art. Generally, nomenclature used in connection with, and techniques of, pharmacology, cell and tissue culture, molecular biology, cell and cancer biology, neurobiology, neurochemistry, virology, immunology, microbiology, genetics and protein and nucleic acid chemistry, described herein, are those well-known and commonly used in the art. In case of conflict, the present specification, including definitions, will control.

The practice of the present application will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Sambrook and Russell, Molecular Cloning: A Laboratory Manual, 3rd. ed., Cold Spring Harbor Laboratory Press, Cold Spring Harbor, N.Y. (2001); Ausubel et al., Current Protocols in Molecular Biology, John Wiley & Sons, N Y (2002); Harlow and Lane Using Antibodies: A Laboratory Manual, Cold Spring Harbor Laboratory Press, Cold Spring Harbor, N.Y. (1998); Coligan et al., Short Protocols in Protein Science, John Wiley & Sons, N Y (2003); Short Protocols in Molecular Biology (Wiley and Sons, 1999).

The nomenclatures used in connection with, and the laboratory procedures and techniques of biochemistry, immunology, microbiology, molecular biology, and virology described herein are those well-known and commonly used in the art.

Throughout this specification and embodiments, the word comprise, or variations such as comprises or comprising, will be understood to imply the inclusion of a stated integer or group of integers but not the exclusion of any other integer or group of integers.

It is understood that wherever embodiments are described herein with the language “comprising,” otherwise analogous embodiments described in terms of “consisting of” and/or “consisting essentially of” are also provided.

The term “including” is used to mean “including but not limited to.” “Including” and “including but not limited to” are used interchangeably.

Any example(s) following the term “e.g.” or “for example” is not meant to be exhaustive or limiting.

Unless otherwise required by context, singular terms shall include pluralities and plural terms shall include the singular.

The articles a, an and “the” are used herein to refer to one or to more than one (i.e., to at least one) of the grammatical object of the article. By way of example, an element means one element or more than one element. Reference to “about” a value or parameter herein includes (and describes) embodiments that are directed to that value or parameter per se. For example, description referring to “about X” includes description of “X.” Numeric ranges are inclusive of the numbers defining the range. As used herein, the term “about” permits a variation of ±10% within the range of the significant digit.

Notwithstanding that the disclosed numerical ranges and parameters are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

Where aspects or embodiments are described in terms of a Markush group or other grouping of alternatives, the present application encompasses not only the entire group listed as a whole, but each member of the group individually and all possible subgroups of the main group, and also the main group absent one or more of the group members. The present application also envisages the explicit exclusion of one or more of any of the group members in the Markush group or other grouping of alternatives.

Exemplary methods and materials are described herein, although methods and materials similar or equivalent to those described herein can also be used in the practice or testing of the various aspects and embodiments. The materials, methods, and examples are illustrative only and not intended to be limiting.

### Definitions

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

As used herein, and unless otherwise specified, the terms “treat”, “treating” and “treatment” and variations thereof refer to an action that occurs in a subject with psoriasis, which reduces the severity of at least one discernible symptom of psoriasis, or retards or slows the progression of at least one discernible symptom of psoriasis. In some embodiments, “treat” and its variations refers to an amelioration of at least one measurable physical parameter, not necessarily discernible by the patient. In some embodiments, “treat” and its variations refers to inhibiting or reducing or slowing the progression of psoriasis, either physically (e.g., stabilization of a discernible symptom), physiologically (e.g., stabilization of a physical parameter), or both, relative to an untreated control. In certain embodiments, “treat” and its variations refers to slowing the progression or reversing the progression of psoriasis relative to an untreated control.

The term “likelihood” generally refers to an increase in the probability of an event. The term “likelihood” when used in reference to the effectiveness of a patient response generally contemplates an increased probability that the symptoms of psoriasis will be lessened or decreased.

As used herein, the term “predict” refers to determine or foresee in advance. When used to “predict” the effectiveness of a psoriasis treatment, for example, the term “predict” can mean that the likelihood of the outcome of the treatment can be determined at the outset, before the treatment has begun, or before the treatment period has progressed substantially.

As used herein, the term “monitor” refers to regularly observing and tracking the progress or quality over a period of time. In the context of this disclosure, “monitoring” may refer to tracking the effectiveness in treating psoriasis in a subject, or a cell or tissue obtained from a subject. Similarly, the term “monitoring,” when used in connection with subject compliance, either individually, or within a clinical trial, refers to the tracking or confirming that the subject is actually following the treatment regimen being tested as prescribed.

As used herein, the term “discriminate” refers to the ability to distinguish psoriasis from other skin conditions, such as atopic dermatitis, based on the biomarker profile obtained from the surface skin samples. In some instances, the presence of NOS2/iNOS in a surface skin sample is indicative of psoriasis and sufficiently discriminates atopic dermatitis from psoriasis. In other instances, a level of NOS2/iNOS above a threshold is indicative of psoriasis and sufficiently discriminates atopic dermatitis from psoriasis. In yet some other instances, a level of NOS2/iNOS above a threshold and levels of one or more biomarkers selected from IL-17C, IL-1B, IL-6, CXCL8/IL-8, DEFB4B, CXCL9, SERPINB3, CCL20, S100A9, IL17A, PI3, CXCL10, and IL36A above a threshold are indicative of psoriasis and sufficiently discriminates atopic dermatitis from psoriasis

As used herein, the term “skin lesion” is a part of the surface skin in a human subject that has an abnormal growth or appearance compared to the skin around it. The skin lesion may be dry, raised, red, and/or covered in silvery white scales. The lesional skin may be patchy, covered in white bumps or pustules. The lesions may appear anywhere on the body, such as the elbows, knees, scalp, lower back, armpits, groin, between the buttocks, under the breasts, etc. The lesional skin may be cracked, itchy, sore and/or bleeding. As used herein, the term “surface skin sample” is one or more samples of surface skin obtained from a subject by a minimally invasive, non-scarring technique such as tape-stripping or equivalent methods. The skin sample may be lesional skin or non-lesional skin from a subject.

In the “tape-stripping” approach, serial adhesive films are used to capture the stratum corneum and the upper part of the granular layer as described previously. See Kim B E, et al. Journal of Investigative Dermatology 2019; 139:2387; Dyjack N, et al. J Allergy Clin Immunol 2018; 141:1298-309. In some instances, at least three tape-strips are obtained from the same skin surface and pooled to obtain a single sample.

“Tape-stripping” or equivalent methods are described in U.S. Pat. No. 7,183,057, incorporated herein in its entirety. The method of tape-stripping involves applying adhesive tape to the skin in a manner sufficient to isolate and remove an epidermal sample adhering to the tape that includes nucleic acid and/or protein molecules. The tape stripping method of the disclosure does not bare the viable epidermis unlike a biopsy which includes cells of the epidermis as well as the upper dermis. Adhesive strips useful for tape-stripping, which need not specifically be in a tape format, include adhesive tapes such as D-squame® and Sebutape™ (polyacrylate ester adhesives; CuDerm Corporation, Dallas, Tex.) or Blenderm™, Tegaderm™, Duct tape (333 Duct Tape, Nashua tape products), Scotch® Tape (3M Scotch 810, St. Paul, Minn.), Diamond™ (The Sellotape Company; Eindhoven, the Netherlands), Sentega™ (polypropylene tape, Sentega Eiketten B V, Utrecht, The Netherlands), hydrogels such as Hypan™ (Hymedix International, Inc., Dayton, N.J.), may be used. The adhesive may be any of the commonly used pressure-sensitive-type adhesives or those which solidify quickly upon skin contact (such as cynaoacrylates). The adhesives may be on flexible or solid backings to make sampling easier. A constant pressure device (e.g. Desquame Pressure Instrument, CuDerm; Dallas, Tex.) can be used to apply pressure to the adhesive device during sampling. Other types of materials with adhesive properties such as glues, gums, and resins may also be used. In some instances, minimally invasive methods equivalent to tape-stripping that do not cause scarring may also be used, such as, mechanical scraping, swabbing and/or direct elution, pressure blotting, electric transfer, or the like.

As used herein, the term “control” refers to a tape-stripped surface skin sample taken from a healthy subject, which skin sample does not exhibit lesions or discoloration. A “reference level” of a biomarker in a control refers to the baseline amount of that biomarker in the tape-stripped normal skin sample. The amount of the biomarker that is measured in the sample may be relative or absolute. In some embodiments the relative expression of mRNA or cDNA is measured in the test sample versus the control sample. In other embodiments, the absolute amount of NOS2/iNOS protein biomarker is measured in the test sample versus the control sample.

As used herein, the term “expressed” or “expression” refers to the transcription from a gene to a ribonucleic acid (RNA) molecule at least complementary in part to a region of one of the two nucleic acid strands of the gene. Alternatively, the term “expressed” or “expression” may refer to the translation from the RNA molecule to give a protein, a polypeptide or a portion thereof.

The level of mRNA or protein expression may “decrease” in a subject administered a psoriatic treatment. Alternatively, the level of mRNA, cDNA or protein may “increase” following psoriasis treatment. In some situations, the mRNA, cDNA or protein level may remain unchanged upon a given treatment, an mRNA, cDNA or protein level can be “downregulated”, i.e., the level of mRNA or protein may be decreased, for example, by about 99%, 95%, 90%, 80%, 70%, 60%, 50%, 40%, 30%, 20%, 10%, 1% or less of the comparative control mRNA, cDNA or protein level. Alternatively, an mRNA, cDNA or protein level from a subject sample can be “upregulated”, i.e., the level of mRNA, cDNA or protein may be increased, for example, by about 5%, 10%, 20%, 30%, 40%, 50%, 60%, 70%, 90%, 100%, 200%, 300%, 500%, 1,000%, 5,000%, 10,000%, 15,000%, 20,000% or more of the comparative control mRNA, cDNA or protein level. In some instances, when the level of NOS2/iNOS mRNA, cDNA or protein in a lesional skin sample is zero or negligible, the subject is determined to have a skin condition other than psoriasis. In other instances, when the level of NOS2/iNOS mRNA, cDNA or protein in a lesional skin sample is not zero or negligible, the subject is determined to have psoriasis but not another skin condition. In some instances, when the absolute amounts of NOS2/iNOS mRNA, cDNA or protein is above a threshold, the subject is determined to have psoriasis. In other instances, when the absolute or relative levels of NOS2/iNOS mRNA, cDNA or protein fall below a threshold level in response to a psoriasis treatment, the subject is said to be responsive to the psoriasis treatment.

### 1. Psoriasis

Psoriasis is a common, chronic, multisystem inflammatory disease with by skin and joint involvement. It is characterized by red, itchy scaly patches, most commonly on the knees, elbows, trunk and scalp. The most common clinical manifestations of psoriasis are plaque psoriasis, guttate psoriasis, inverse psoriasis, pustular psoriasis, erythrodermic psoriasis, and annular psoriasis.

Plaque psoriasis is the most common form, plaque psoriasis causes dry, raised, red skin patches (lesions) covered with silvery scales. The plaques might be itchy or tender, and there may be few or many. They usually appear on elbows, knees, lower back and scalp. Nail psoriasis affects fingernails and toenails, causing pitting, abnormal nail growth and discoloration. Psoriatic nails might loosen and separate from the nail bed (onycholysis). Guttate psoriasis primarily affects young adults and children. It is usually triggered by a bacterial infection such as strep throat. It is marked by small, drop-shaped, scaling lesions on the trunk, arms or legs. Inverse psoriasis mainly affects the skin folds of the groin, buttocks and breasts and causes smooth patches of red skin that worsen with friction and sweating. Fungal infections may trigger this type of psoriasis. Pustular psoriasis is a rare form of psoriasis which causes clearly defined pus-filled lesions that occur in widespread patches (generalized pustular psoriasis) or in smaller areas on the palms of the hands or the soles of the feet. Erythrodermic psoriasis is the least common type of psoriasis, and can cover the entire body with a red, peeling rash that can itch or burn intensely. Psoriatic arthritis can appear on the scalp, face, hands, feet, nails, genitals, and in skin folds, such as the armpits and under the breasts, and causes swollen, painful joints that are typical of arthritis. It can cause stiffness and progressive joint damage that in the most serious cases may lead to permanent joint damage.

Psoriasis often develops between the ages of 15 and 35, but it can develop at any age. About 10 to 15 percent of those with psoriasis get it before age 10. Some infants have psoriasis, although this is considered rare. Pathogenesis is multifactorial, involving dysregulated inflammation and genetic associations. Beyond the physical dimensions of disease, psoriasis has an extensive emotional and psychosocial effect on patients; it can result in stigmatization, poor self-esteem, and increased stress, affecting social functioning and interpersonal relationships. See Kim W et al Canadian Family Physician 63: April (2017):278.

The psoriasis can be mild, moderate or severe. Mild psoriasis covers less than 3% of a patient's body surface area (BSA) and can be controlled by routine skin case measures and topical therapy. Moderate psoriasis covers 3-10% BSA and affects the patient's quality of life either due to the extent of the disease, physical discomfort (pain or pruritis), or location (e.g., the face, hands, feet, or genitals). Severe psoriasis covers>10% BSA cannot or would not be expected to be satisfactorily controlled by topical therapy and causes severe degradation of the patient's quality of life.

The hallmark of psoriasis is sustained inflammation that leads to uncontrolled keratinocyte proliferation and dysfunctional differentiation. The histology of the psoriatic plaque shows acanthosis (epidermal hyperplasia), which overlies inflammatory infiltrates composed of dermal dendritic cells, macrophages, T cells, and neutrophils. Neovascularization is also a prominent feature. See Rendon A and Schakel K Int J of Mol Sci (2019); 20:1475.

### 2. Atopic Dermatitis

Atopic dermatitis (AD) is a condition that causes dry, itchy and inflamed skin. It can be common in young children but can occur at any age. Atopic dermatitis can be long lasting (chronic) and can exhibit episodic recurrences. AD can be irritating but is not contagious. Symptoms of AD can include dry cracked skin; itchiness; rash on swollen skin that varies in color depending on skin color; small, raised bumps on the skin; oozing and crusting; thickened skin; darkening of the skin around the eyes; and raw, sensitive skin from scratching. AD onset can begin before age 5 and may continue into the teen and adult years. For some people, AD symptoms flare and then clear up for a time, even for several years.

### 3. Therapies for Psoriasis

Treatment of psoriasis can vary depending on the severity of the disease. Mild to moderate psoriasis can be treated topically with a combination of glucocorticoids, vitamin D analogues, and phototherapy. Moderate to severe psoriasis often requires systemic treatment. The presence of comorbidities such as psoriasis arthritis is also highly relevant in treatment selection.

In some instances, psoriasis treatment involves treating the disease topically with a cream, lotion, spray, moisturizer, bath salt, immunomodulator, coal tar, anthralin, corticosteroid, and/or vitamin D analogue. The moisturizer may contain one or more of salicylic acid, lactic acid, petroleum jelly, and paraffin. In some instances, the psoriatic therapy involves treatment with a small-molecule therapeutic or targeted biological drug. Examples of small-molecule therapeutics include but are not limited to methotrexate, cyclosporine, acitretin, retinoids, fumaric acid esters, apremilast. Immunomodulators including but not limited to tacrolimis and pimecrolimus may also be used to treat psoriasis. Biological drugs including but not limited to Abatacept (Orencia®), adalimumab (Humira®), certolizumab (Cimzia®), brodalumab (Silig™) dupilumab (Dupixent®), etanercept (Enbrel®), golimumab (Simponi®), guselkumab (Tremfya®), infliximab (Avsola™), ixekizumab (Taltz), risankizumab (Skyrizi™), secukinumab (Cosentyx®), tildrakizumab (Ilumya™), and ustekinumab (Stelara®) may also be used to treat psoriasis. The drug may be administered orally, subcutaneously or systemically.

### 4. Therapies for Atopic Dermatitis

In some instances, AD treatment involves treating the disease topically with a cream, lotion, spray, moisturizer, bath salt, or immunomodulator. In some instances, AD treatment includes at least one therapy selected from a group consisting of topical treatment, phototherapy, oral medication, and a biological drug. In some instances, the biological drug is selected from dupilumab, dupixent, traloknumab, upadicitinib, abrocotinib, and lebrikizumab. In some instances, the topical treatment is ruxolitinib.

### 5. Biomarkers for Psoriasis

In order to diagnose subjects with psoriasis, identify subjects who would benefit from treatment with a psoriasis therapy (such as the one or more of the psoriasis therapies described above), distinguish psoriasis from other skin conditions such as AD, or to determine if a therapy is working, it is helpful to have a biomarker. A biomarker is a characteristic that can be objectively measured and evaluated as an indicator of normal biologic processes, pathologic processes, or pharmacological responses to a therapeutic intervention. Biomarkers can be biological (e.g., small molecules, metabolites, peptides, proteins, RNA, DNA), physiological (e.g., blood pressure, electromyography, respiratory function), or structural measures (e.g., ultrasound, magnetic resonance imaging, or histological assessment). The biomarkers may be prognostic biomarkers that predict a future clinical outcome; disease progression biomarkers that are indicative of the severity of disease impact; predictive biomarkers that predict a future clinical response to therapy and helps stratify therapies; pharmacodynamics biomarkers that monitor or quantify a therapeutic effect; and surrogate end point biomarkers that predict a future clinical response to therapy wherein a change in the end point is associated with a future clinical response.

There are several known biomarkers for psoriasis. See e.g., Tampa A et al, Dis Markers. 2018: 5823684, which include soluble biomarkers, tissue-associated biomarkers, psychopathology biomarkers, oxidative stress biomarkers, etc. Such biomarkers include C-Reactive Protein, connexins, VEGF, TGF-β1, HBD-2, and IL-18, etc. However, to date, there is no ideal biomarker for psoriasis.

This disclosure illustrates the use of NOS2/iNOS as a highly effective biomarker as measured in a non-invasively obtained skin sample. NOS2/iNOS was identified as a single gene classifier, able to discriminate lesional AD from lesional psoriasis with 100% accuracy.

NOS2 encodes for the inducible nitric oxidase synthase which catalyzes the production of nitric oxide (NO) and plays an important role in metabolic and inflammatory processes. It has been shown that disease activity of psoriasis correlates with dendritic cells expressing TNF-α and NOS2. See Garzorz N and Eyerich K Expert Review of Clinical Immunology 2015 (11)(2):167-169.

The nucleic acid and amino acid sequences of human NOS2 are provided in SEQ ID NO: 1 and SEQ ID NO: 2 respectively.

SEQ ID NO: 1: human NOS2 nucleic acid sequence
ATAACTTTGTAGCGAGTCGAAAACTGAGGCTCCGGCCGCAGAGAACTCAGCCTCATTCCTGCTTTAAAATCTCTCGGCCACCT
TTGATGAGGGGACTGGGCAGTTCTAGACAGTCCCGAAGTTCTCAAGGCACAGGTCTCTTCCTGGTTTGACTGTCCTTACCCCG
GGGAGGCAGTGCAGCCAGCTGCAAGCCCCACAGTGAAGAACATCTGAGCTCAAATCCAGATAAGTGACATAAGTGACCTGCTT
TGTAAAGCCATAGAGATGGCCTGTCCTTGGAAATTTCTGTTCAAGACCAAATTCCACCAGTATGCAATGAATGGGGAAAAAGA
CATCAACAACAATGTGGAGAAAGCCCCCTGTGCCACCTCCAGTCCAGTGACACAGGATGACCTTCAGTATCACAACCTCAGCA
AGCAGCAGAATGAGTCCCCGCAGCCCCTCGTGGAGACGGGAAAGAAGTCTCCAGAATCTCTGGTCAAGCTGGATGCAACCCCA
TTGTCCTCCCCACGGCATGTGAGGATCAAAAACTGGGGCAGCGGGATGACTTTCCAAGACACACTTCACCATAAGGCCAAAGG
GATTTTAACTTGCAGGTCCAAATCTTGCCTGGGGTCCATTATGACTCCCAAAAGTTTGACCAGAGGACCCAGGGACAAGCCTA
CCCCTCCAGATGAGCTTCTACCTCAAGCTATCGAATTTGTCAACCAATATTACGGCTCCTTCAAAGAGGCAAAAATAGAGGAA
CATCTGGCCAGGGTGGAAGCGGTAACAAAGGAGATAGAAACAACAGGAACCTACCAACTGACGGGAGATGAGCTCATCTTCGC
CACCAAGCAGGCCTGGCGCAATGCCCCACGCTGCATTGGGAGGATCCAGTGGTCCAACCTGCAGGTCTTCGATGCCCGCAGCT
GTTCCACTGCCCGGGAAATGTTTGAACACATCTGCAGACACGTGCGTTACTCCACCAACAATGGCAACATCAGGTCGGCCATC
ACCGTGTTCCCCCAGCGGAGTGATGGCAAGCACGACTTCCGGGTGTGGAATGCTCAGCTCATCCGCTATGCTGGCTACCAGAT
GCCAGATGGCAGCATCAGAGGGGACCCTGCCAACGTGGAATTCACTCAGCTGTGCATCGACCTGGGCTGGAAGCCCAAGTACG
GCCGCTTCGATGTGGTCCCCCTGGTCCTGCAGGCCAATGGCCGTGACCCTGAGCTCTTCGAAATCCCACCTGACCTTGTGCTT
GAGGTGGCCATGGAACATCCCAAATACGAGTGGTTTCGGGAACTGGAGCTAAAGTGGTACGCCCTGCCTGCAGTGGCCAACAT
GCTGCTTGAGGTGGGCGGCCTGGAGTTCCCAGGGTGCCCCTTCAATGGCTGGTACATGGGCACAGAGATCGGAGTCCGGGACT
TCTGTGACGTCCAGCGCTACAACATCCTGGAGGAAGTGGGCAGGAGAATGGGCCTGGAAACGCACAAGCTGGCCTCGCTCTGG
AAAGACCAGGCTGTCGTTGAGATCAACATTGCTGTGCTCCATAGTTTCCAGAAGCAGAATGTGACCATCATGGACCACCACTC
GGCTGCAGAATCCTTCATGAAGTACATGCAGAATGAATACCGGTCCCGTGGGGGCTGCCCGGCAGACTGGATTTGGCTGGTCC
CTCCCATGTCTGGGAGCATCACCCCCGTGTTTCACCAGGAGATGCTGAACTACGTCCTGTCCCCTTTCTACTACTATCAGGTA
GAGGCCTGGAAAACCCATGTCTGGCAGGACGAGAAGCGGAGACCCAAGAGAAGAGAGATTCCATTGAAAGTCTTGGTCAAAGC
TGTGCTCTTTGCCTGTATGCTGATGCGCAAGACAATGGCGTCCCGAGTCAGAGTCACCATCCTCTTTGCGACAGAGACAGGAA
AATCAGAGGCGCTGGCCTGGGACCTGGGGGCCTTATTCAGCTGTGCCTTCAACCCCAAGGTTGTCTGCATGGATAAGTACAGG
CTGAGCTGCCTGGAGGAGGAACGGCTGCTGTTGGTGGTGACCAGTACGTTTGGCAATGGAGACTGCCCTGGCAATGGAGAGAA
ACTGAAGAAATCGCTCTTCATGCTGAAAGAGCTCAACAACAAATTCAGGTACGCTGTGTTTGGCCTCGGCTCCAGCATGTACC
CTCGGTTCTGCGCCTTTGCTCATGACATTGATCAGAAGCTGTCCCACCTGGGGGCCTCTCAGCTCACCCCGATGGGAGAAGGG
GATGAGCTCAGTGGGCAGGAGGACGCCTTCCGCAGCTGGGCCGTGCAAACCTTCAAGGCAGCCTGTGAGACGTTTGATGTCCG
AGGCAAACAGCACATTCAGATCCCCAAGCTCTACACCTCCAATGTGACCTGGGACCCGCACCACTACAGGCTCGTGCAGGACT
CACAGCCTTTGGACCTCAGCAAAGCCCTCAGCAGCATGCATGCCAAGAACGTGTTCACCATGAGGCTCAAATCTCGGCAGAAT
CTACAAAGTCCGACATCCAGCCGTGCCACCATCCTGGTGGAACTCTCCTGTGAGGATGGCCAAGGCCTGAACTACCTGCCGGG
GGAGCACCTTGGGGTTTGCCCAGGCAACCAGCCGGCCCTGGTCCAAGGTATCCTGGAGCGAGTGGTGGATGGCCCCACACCCC
ACCAGACAGTGCGCCTGGAGGCCCTGGATGAGAGTGGCAGCTACTGGGTCAGTGACAAGAGGCTGCCCCCCTGCTCACTCAGC
CAGGCCCTCACCTACTTCCTGGACATCACCACACCCCCAACCCAGCTGCTGCTCCAAAAGCTGGCCCAGGTGGCCACAGAAGA
GCCTGAGAGACAGAGGCTGGAGGCCCTGTGCCAGCCCTCAGAGTACAGCAAGTGGAAGTTCACCAACAGCCCCACATTCCTGG
AGGTGCTAGAGGAGTTCCCGTCCCTGCGGGTGTCTGCTGGCTTCCTGCTTTCCCAGCTCCCCATTCTGAAGCCCAGGTTCTAC
TCCATCAGCTCCTCCCGGGATCACACGCCCACAGAGATCCACCTGACTGTGGCCGTGGTCACCTACCACACCCGAGATGGCCA
GGGTCCCCTGCACCACGGCGTCTGCAGCACATGGCTCAACAGCCTGAAGCCCCAAGACCCAGTGCCCTGCTTTGTGCGGAATG
CCAGCGGCTTCCACCTCCCCGAGGATCCCTCCCATCCTTGCATCCTCATCGGGCCTGGCACAGGCATCGCGCCCTTCCGCAGT
TTCTGGCAGCAACGGCTCCATGACTCCCAGCACAAGGGAGTGCGGGGAGGCCGCATGACCTTGGTGTTTGGGTGCCGCCGCCC
AGATGAGGACCACATCTACCAGGAGGAGATGCTGGAGATGGCCCAGAAGGGGGTGCTGCATGCGGTGCACACAGCCTATTCCC
GCCTGCCTGGCAAGCCCAAGGTCTATGTTCAGGACATCCTGCGGCAGCAGCTGGCCAGCGAGGTGCTCCGTGTGCTCCACAAG
GAGCCAGGCCACCTCTATGTTTGCGGGGATGTGCGCATGGCCCGGGACGTGGCCCACACCCTGAAGCAGCTGGTGGCTGCCAA
GCTGAAATTGAATGAGGAGCAGGTCGAGGACTATTTCTTTCAGCTCAAGAGCCAGAAGCGCTATCACGAAGATATCTTTGGTG
CTGTATTTCCTTACGAGGCGAAGAAGGACAGGGTGGCGGTGCAGCCCAGCAGCCTGGAGATGTCAGCGCTCTGAGGGCCTACA
GGAGGGGTTAAAGCTGCCGGCACAGAACTTAAGGATGGAGCCAGCTCTGCATTATCTGAGGTCACAGGGCCTGGGGAGATGGA
GGAAAGTGATATCCCCCAGCCTCAAGTCTTATTTCCTCAACGTTGCTCCCCATCAAGCCCTTTACTTGACCTCCTAACAAGTA
GCACCCTGGATTGATCGGAGCCTCCTCTCTCAAACTGGGGCCTCCCTGGTCCCTTGGAGACAAAATCTTAAATGCCAGGCCTG
GCAAGTGGGTGAAAGATGGAACTTGCTGCTGAGTGCACCACTTCAAGTGACCACCAGGAGGTGCTATCGCACCACTGTGTATT
TAACTGCCTTGTGTACAGTTATTTATGCCTCTGTATTTAAAAAACTAACACCCAGTCTGTTCCCCATGGCCACTTGGGTCTTC
CCTGTATGATTCCTTGATGGAGATATTTACATGAATTGCATTTTACTTTAATCACA
SEQ ID NO: 2: human NOS2 amino acid sequence
MACPWKFLFKTKFHQYAMNGEKDINNNVEKAPCATSSPVTQDDLQYHNLSKQQNESPQPLVETGKKSPESLVKLDATPLSSPR
HVRIKNWGSGMTFQDTLHHKAKGILTCRSKSCLGSIMTPKSLTRGPRDKPTPPDELLPQAIEFVNQYYGSFKEAKIEEHLARV
EAVTKEIETTGTYQLTGDELIFATKQAWRNAPRCIGRIQWSNLQVFDARSCSTAREMFEHICRHVRYSTNNGNIRSAITVFPQ
RSDGKHDFRVWNAQLIRYAGYQMPDGSIRGDPANVEFTQLCIDLGWKPKYGRFDVVPLVLQANGRDPELFEIPPDLVLEVAME
HPKYEWFRELELKWYALPAVANMLLEVGGLEFPGCPFNGWYMGTEIGVRDFCDVQRYNILEEVGRRMGLETHKLASLWKDQAV
VEINIAVLHSFQKQNVTIMDHHSAAESFMKYMQNEYRSRGGCPADWIWLVPPMSGSITPVFHQEMLNYVLSPFYYYQVEAWKT
HVWQDEKRRPKRREIPLKVLVKAVLFACMLMRKTMASRVRVTILFATETGKSEALAWDLGALFSCAFNPKVVCMDKYRLSCLE
EERLLLVVTSTFGNGDCPGNGEKLKKSLFMLKELNNKFRYAVFGLGSSMYPRFCAFAHDIDQKLSHLGASQLTPMGEGDELSG
QEDAFRSWAVQTFKAACETFDVRGKQHIQIPKLYTSNVTWDPHHYRLVQDSQPLDLSKALSSMHAKNVFTMRLKSRQNLQSPT
SSRATILVELSCEDGQGLNYLPGEHLGVCPGNQPALVQGILERVVDGPTPHQTVRLEALDESGSYWVSDKRLPPCSLSQALTY
FLDITTPPTQLLLQKLAQVATEEPERQRLEALCQPSEYSKWKFTNSPTFLEVLEEFPSLRVSAGFLLSQLPILKPRFYSISSS
RDHTPTEIHLTVAVVTYHTRDGQGPLHHGVCSTWLNSLKPQDPVPCFVRNASGFHLPEDPSHPCILIGPGTGIAPFRSFWQQR
LHDSQHKGVRGGRMTLVFGCRRPDEDHIYQEEMLEMAQKGVLHAVHTAYSRLPGKPKVYVQDILRQQLASEVLRVLHKEPGHL
YVCGDVRMARDVAHTLKQLVAAKLKLNEEQVEDYFFQLKSQKRYHEDIFGAVFPYEAKKDRVAVQPSSLEMSAL

In certain instances, the level of NOS2/iNOS is used in combination with the levels of one or more other biomarkers to diagnose psoriasis, monitor treatment efficacy, or differentiate psoriasis from other skin conditions. These biomarkers include but are not limited to IL-17C, IL-1β, IL-6, CXCL8/IL-8, TNFα, IFNγ, CXCL9, CXCL10, IL-17A, IL-17F, IL-23p19, IL-12/IL-23p40, IL-36A, IL-36G, DEFB4B, CCL20, CXCL1, CAMP/LL37, PI3, SERPINB3, STAT3, S100A9, PI3, and S100A12.

### 6. Diagnosing Psoriasis

This disclosure features methods of diagnosing whether a subject has biologically active disease (i.e., whether the psoriasis is active and its severity). The method involves measuring a biomarker level (i.e., NOS2/iNOS level) in a surface skin sample obtained from the subject, more particularly the surface skin sample is a surface skin sample from a subject by a non-invasive non-scarring technique such as tape-stripping. In some instances, psoriasis is diagnosed if the NOS2/iNOS level in the subject is higher than a control level. In other instances, psoriasis is diagnosed if the NOS2/iNOS is present in the subject in a non-zero amount. The NOS2/iNOS level also predicts the severity of the disease: i.e., the higher the NOS2/iNOS level relative to a control, the more severe the psoriasis.

In certain instances, the subject is a human who is greater than 18 months of age. In some instances, the subject is a human who is greater than 18 years of age.

In some instances, the NOS2/iNOS level is measured by assessing the level of NOS2/iNOS RNA (e.g., mRNA or cDNA) in the surface skin sample.

In some instances, the NOS2/iNOS level is measured by assessing the level of an NOS2/iNOS protein in the surface skin sample. The concentration of the protein or proteins of interest can be measured using any method known in the art such as an immunological assay. Non-limiting examples of such methods include enzyme immunoassay, radioimmunoassay, chemiluminescent immunoassay, electrochemiluminescence immunoassay, latex turbidimetric immunoassay, latex photometric immunoassay, immuno-chromatographic assay, and western blotting. In certain embodiments, the concentration of the protein or proteins of interest is measured by mass spectrometry.

In some embodiments, the presence of NOS2/iNOS in the surface skin sample is indicative of psoriasis. In other embodiments, the NOS2/iNOS in the surface skin sample above a control or threshold level is indicative of psoriasis.

A human subject who is diagnosed as having psoriasis can be administered any psoriasis therapy. In certain cases, a human subject who is previously determined to have psoriasis (e.g by measuring NOS2/iNOS levels in a surface skin sample from the subject) is administered one or more of the psoriasis therapies described earlier.

### 7. Responsiveness to Treatment

The levels of NOS2/iNOS can also be used to determine if a subject receiving a psoriasis therapy is responding to the treatment. This can be assessed by obtaining a first surface skin sample from the subject before and a second surface skin sample after administering one or more psoriasis therapies to the subject and measuring the level of NOS2/iNOS in such samples.

In certain instances, the first surface skin sample or samples can be collected from the subject any time before treatment, e.g., a week before, several days before, a day before, several hours before, an hour before, or less than an hour before, administering the psoriasis therapy. Similarly, the second surface skin sample or samples can be collected from the subject any time after administration of the psoriasis treatment, e.g., less than an hour after, an hour after, several hours after, a day after, several days after, a week after, several weeks after, a month after, two months after, three months after, four months after, five months after, 6 months after, 7 months after, or 8 months after, administering the psoriasis therapy. A reduction in NOS2/iNOS level after commencing psoriasis therapy is indicative of the effectiveness of the psoriasis therapy. In such instances, continuation of the psoriasis therapy is indicated. Failure to reduce NOS2/iNOS level after commencing a psoriasis therapy is indicative of the need for altering the dose (e.g., increasing the dose) of the psoriasis therapy, or the lack of effectiveness of that particular psoriasis therapy. In the latter instance, discontinuation of that particular psoriasis therapy may be suggested and the use of a different psoriasis therapy or therapies is to be considered.

The level of NOS2/iNOS can be assessed by measuring RNA, cDNA, or protein levels. In some instances, the absolute or relative expression level of NOS2/iNOS mRNA is determined. A variety of suitable methods can be employed to detect and/or measure the level of mRNA expression of a gene. For example, mRNA expression can be determined using Northern blot or dot blot analysis, reverse transcriptase-PCR (RT-PCR; e.g., quantitative RT-PCR), in situ hybridization (e.g., quantitative in situ hybridization) or nucleic acid array (e.g., oligonucleotide arrays or gene chips) analysis, and the like. Details of such methods are described in, e.g., Sambrook et al, Molecular Cloning: A Laboratory Manual Second Edition vol. 1, 2 and 3. Cold Spring Harbor Laboratory Press: Cold Spring Harbor, N.Y., USA, November 1989; Gibson et al. (1999) Genome Res., 6(10):995-1001; and Zhang et al. (2005) Environ. Sci. Technol., 39(8):2777-2785; U.S. Publication No. 2004086915; European Patent No. 0543942; and U.S. Pat. No. 7,101,663; the disclosures of each of which are incorporated herein by reference in their entirety. When the NOS2/iNOS biomarker is an mRNA molecule, the mRNA sequence, or a fragment thereof, can be used to prepare a probe that is at least partially complementary. The probe can then be used to detect the mRNA sequence in a sample, using any suitable assay, such as PCR-based methods, Northern blotting, a dipstick assay, and the like.

The concentration of the NOS2/iNOS protein can be measured using any method known in the art such as an immunological assay. Non-limiting examples of such methods include enzyme immunoassay, radioimmunoassay, chemiluminescent immunoassay, electrochemiluminescence immunoassay, latex turbidimetric immunoassay, latex photometric immunoassay, immunochromatographic assay, and western blotting. In certain embodiments, the concentration of the protein or proteins of interest is measured by mass spectrometry.

In certain instances, an NOS2/iNOS mRNA, cDNA or protein level in the first surface skin sample is a non-zero amount. In certain embodiments, the NOS2/iNOS mRNA, cDNA or protein level measured in the second surface skin sample as obtained at the same location as the first surface skin sample is lower than the NOS2/iNOS mRNA, cDNA or protein level measured in the first surface skin sample. In some embodiments, the NOS2/iNOS mRNA, cDNA or protein level measured in the second surface skin sample shows a greater than 30% (e.g., greater than 31%, 35%, 40%, 45%, 50%, 55%, 60%, 65%, 70%, 80%, 85%, 90%, or 95%) decline relative to the NOS2/iNOS mRNA, cDNA or protein level measured in the first surface skin sample. In some embodiments, the NOS2/iNOS mRNA, cDNA or protein level measured in the second surface skin sample is between 10% to 80% of the NOS2/iNOS mRNA, cDNA or protein level measured in the first surface skin sample. In some embodiments, the NOS2/iNOS mRNA, cDNA or protein level measured in the second surface skin sample is between 20% to 80% of the NOS2/iNOS mRNA, cDNA or protein level measured in the first surface skin sample. In some embodiments, the NOS2/iNOS mRNA, cDNA or protein level measured in the second surface skin sample is between 20% to 85% of the NOS2/iNOS mRNA, cDNA or protein level measured in the first surface skin sample. In some embodiments, the NOS2/iNOS mRNA, cDNA or protein level measured in the second surface skin sample is between 20% to 90% of the NOS2/iNOS mRNA, cDNA or protein level measured in the first surface skin sample. In some embodiments, the NOS2/iNOS mRNA, cDNA or protein level measured in the second surface skin sample is between 20% to 95% of the NOS2/iNOS mRNA, cDNA or protein level measured in the first surface skin sample. In some embodiments, the NOS2/iNOS mRNA, cDNA or protein level measured in the second surface skin sample is between 30% to 80% of the NOS2/iNOS mRNA, cDNA or protein level measured in the first surface skin sample. In some embodiments, the NOS2/iNOS mRNA, cDNA or protein level measured in the second surface skin sample is between 30% to 85% of the NOS2/iNOS mRNA, cDNA or protein level measured in the first surface skin sample. In some embodiments, the NOS2/iNOS mRNA, cDNA or protein level measured in the second surface skin sample is between 30% to 90% of the NOS2/iNOS mRNA, cDNA or protein level measured in the first surface skin sample. In some embodiments, the NOS2/iNOS mRNA, cDNA or protein level measured in the second surface skin sample is between 30% to 95% of the NOS2/iNOS mRNA, cDNA or protein level measured in the first surface skin sample.

In certain instances, the subject is a human who is greater than 18 months of age. In some instances, the subject is a human who is greater than 18 years of age. The levels of NOS2/iNOS in a surface skin sample from a subject can also serve to monitor a subject's response to psoriasis treatment.

### 8. Controls

As described above, the methods of the present disclosure can involve, measuring the expression level (e.g., mRNA, cDNA, or protein concentration) of NOS2/iNOS in a surface skin sample from a subject (e.g., a human subject with a skin lesion), wherein the expression level of NOS2/iNOS gene or protein, compared to a control, predicts whether a subject has psoriasis; the severity of the psoriasis; whether or not a subject is a responder to treatment comprising a psoriasis therapy, and monitoring subject response to psoriasis treatment.

In certain embodiments, when diagnosing whether a subject has psoriasis, where the concentration of a NOS2/iNOS in a surface skin sample from a subject is higher than the control, the subject is identified as likely to have psoriasis. In this context, the term “control” includes a sample (from the same source—i.e., surface skin) obtained from a subject of the same or similar age who is known to not have psoriasis. For example, if a subject who is 25 years of age is being tested, then the control is also from a subject who is 25 years of age who do not have psoriasis. The term “control” also includes a sample (from the same tissue) obtained in the past from a subject who is known to not have psoriasis and used as a reference for future comparisons to test samples taken from subjects for whom psoriasis is to be predicted. The “control” expression level/concentration for a NOS2/iNOS mRNA, cDNA or protein may also be pre-established by an analysis of mRNA, cDNA, or protein expression in one or more (e.g., two, three, four, five, six, seven, eight, nine, 10, 15, 20, 25, 30, 35, or 40 or more) human subjects of similar age that do not have psoriasis. This pre-established reference value (which may be an average or median expression level/concentration taken from multiple subjects that do not have psoriasis) may then be used for the “control” concentration/expression level of the protein or nucleic acid in the comparison with the test sample. In such a comparison, the subject is predicted to have psoriasis if the expression level of the NOS2/iNOS being analyzed is higher than the pre-established reference.

In certain embodiments, the “control” is a pre-determined cut-off value. In some embodiments, the methods described herein include determining if the concentration of NOS2/iNOS biomarker falls above or below a predetermined cut-off value. A cut-off value is typically a concentration of a protein above or below which is considered predictive of something—e.g., likely to develop psoriasis; or responsiveness of a subject to a therapy of interest. Thus, in accordance with the methods described herein, a reference concentration of NOS2/iNOS mRNA, cDNA or protein is identified as a cut-off value, above or below of which is predictive of a subject having psoriasis, or of a subject who shows responsiveness to a psoriasis therapy. Some cut-off values are not absolute in that clinical correlations can still remain significant over a range of values on either side of the cutoff; however, it is possible to select an optimal cut-off value (e.g. varying H-scores) of concentration of NOS2/iNOS mRNA, cDNA or protein for a particular sample type. Cut-off values determined for use in the methods described herein can be compared with, e.g., published ranges of NOS2/iNOS concentrations, but can be individualized to the methodology used and patient population. It is understood that improvements in optimal cut-off values could be determined depending on the sophistication of statistical methods used and on the number and source of samples used to determine reference level values for the different proteins, genes, and sample types. Therefore, established cut-off values can be adjusted up or down, on the basis of periodic reevaluations or changes in methodology or population distribution.

The reference concentration of one or more biomarkers of the present disclosure (e.g., NOS2/iNOS) can be determined by a variety of methods. For instance, the reference level can be determined by comparison of the concentration of NOS2/iNOS protein of interest in, e.g., populations of subjects (e.g., patients) that are responsive to a psoriatic therapy or not responsive to a psoriatic therapy. This can be accomplished, for example, by histogram analysis, in which an entire cohort of patients is graphically presented, wherein a first axis represents the concentration of a protein of interest and a second axis represents the number of subjects in the cohort whose sample contain one or more concentrations. Determination of the reference concentration of a protein can then be made based on an amount or concentration which best distinguishes these separate groups. The reference level can be a single number, equally applicable to every subject, or the reference level can vary, according to specific subpopulations of subjects. For example, older subjects can have a different reference level than younger subjects. In addition, a subject with more severe disease can have a different reference value than one with a milder form of the disease (e.g., mild vs severe psoriasis).

The pre-established cut-off value can be an NOS2/iNOS protein concentration that is determined based on receiver operating characteristic (ROC) analysis. ROC curves are used to determine a cut-off value for a clinical test. Consider the situation where there are two groups of patients and by using an established standard technique one group is known to be responsive to a psoriasis therapy, and the other is known to not respond to the psoriasis therapy. A measurement using a surface skin sample from all members of the two groups is used to test for responsiveness to a psoriasis therapy. The test will find some, but not all, responders to respond to a psoriasis therapy. The ratio of the responders found by the test to the total number of responders (known by the established standard technique) is the true positive rate (also known as sensitivity). The test will find some, but not all, non-responders to not respond to a psoriasis therapy. The ratio of the non-responders found by the test to the total number of non-responders (known by the established standard technique) is the true negative rate (also known as specificity). The hope is that the ROC curve analysis of the psoriasis therapy responsiveness test will find a cut-off value that will minimize the number of false positives and false negatives. A ROC is a graphical plot which illustrates the performance of a binary class stratifier system as its discrimination threshold is varied. It is created by plotting the fraction of true positives out of the positives versus the fraction of false positives out of the negatives, at various threshold settings.

In one embodiment, the NOS2/iNOS protein concentration is determined based on ROC analysis predicting response to a psoriasis therapy with a positive predictive value, wherein a concentration of a protein of interest (e.g., NOS2/iNOS) equal to or below the pre-established cut off value is a low concentration of the protein of interest and a value higher than the pre-established cut-off value is a high concentration of the protein of interest. The positive predictive value is the proportion of positive test results that are true positives; it reflects the probability that a positive test reflects the underlying condition being tested for. Methods of constructing ROC curves and determining positive predictive values are well known in the art.

In another embodiment, the pre-established cut-off value can be an NOS2/iNOS protein concentration that is determined based on simulation models predicting responsiveness to psoriasis therapy, and wherein a concentration of the protein of interest equal to or below the pre-established cut-off value is a low concentration of the protein of interest and a value higher than the pre-established cut-off value is a high concentration of the protein of interest.

### 9. Determining Expression Levels/Concentrations of Biomarkers

Gene expression can be detected as, e.g., protein or RNA expression of a target gene. That is, the presence or expression level (amount) of a gene can be determined by detecting and/or measuring the level of mRNA or protein expression of the gene. In some embodiments, gene expression can be detected as the activity of a protein encoded by NOS2/iNOS gene.

In one embodiment, the expression of a gene can be determined by detecting and/or measuring expression or concentration of a protein encoded by the gene. Methods of determining protein expression/concentration are well known in the art. A generally used method involves the use of antibodies specific for the target protein of interest. For example, methods of determining protein expression include, but are not limited to, western blot or dot blot analysis, immunohistochemistry (e.g., quantitative immunohistochemistry), immunocytochemistry, enzyme-linked immunosorbent assay (ELISA), enzyme-linked immunosorbent spot (ELISPOT; Coligan, J. E., et al., eds. (1995) Current Protocols in Immunology. Wiley, New York), radioimmunoassay, chemiluminescent immunoassay, electrochemiluminescence immunoassay, latex turbidimetric immunoassay, latex photometric immunoassay, immuno-chromatographic assay, and antibody array analysis (see, e.g., U.S. Publication Nos. 2003/0013208 and 2004/171068, the disclosures of each of which are incorporated herein by reference in their entirety). Further description of many of the methods above and additional methods for detecting protein expression can be found in, e.g., Sambrook et al. (supra).

In one example, the presence or amount of NOS2/iNOS protein expression of NOS2/iNOS gene can be determined using a western blotting technique. For example, a lysate can be prepared from a surface skin sample, or the surface skin sample itself, can be contacted with Laemmli buffer and subjected to sodium-dodecyl sulfate polyacrylamide gel electrophoresis (SDS-PAGE). SDS-PAGE-resolved proteins, separated by size, can then be transferred to a filter membrane (e.g., nitrocellulose) and subjected to immunoblotting techniques using a detectably-labeled antibody specific to the protein of interest. The presence or amount of bound detectably-labeled antibody indicates the presence or amount of protein in the surface skin sample.

In one embodiment, the SimplePlex platform is used to measure the levels of NOS2/iNOS. SimplePlex is commercially available from Protein Simple (San Jose, Calif., USA) (See Dysinger M, et al. J. Immunol. Methods. 451:1-10, 2017).

In another example, an immunoassay can be used for detecting and/or measuring the protein expression of a gene (e.g., NOS2/iNOS gene). As above, for the purposes of detection, an immunoassay can be performed with an antibody that bears a detection moiety (e.g., a fluorescent agent or enzyme). Proteins from a surface skin sample can be conjugated directly to a solid-phase matrix (e.g., a multi-well assay plate, nitrocellulose, agarose, sepharose, encoded particles, or magnetic beads) or it can be conjugated to a first member of a specific binding pair (e.g., biotin or streptavidin) that attaches to a solid-phase matrix upon binding to a second member of the specific binding pair (e.g., streptavidin or biotin). Such attachment to a solid-phase matrix allows the proteins to be purified away from other interfering or irrelevant components of the surface skin sample prior to contact with the detection antibody and also allows for subsequent washing of unbound antibody. Here, as above, the presence or amount of bound detectably-labeled antibody indicates the presence or amount of protein in the surface skin sample.

There is no particular restriction as to the form of the antibody and the present disclosure includes polyclonal antibodies, as well as monoclonal antibodies. The antiserum obtained by immunizing animals, such as rabbits with a protein or fragment thereof of the invention (i.e., a protein or an immunological fragment thereof of NOS2/iNOS protein), as well polyclonal and monoclonal antibodies of all classes, human antibodies, and humanized antibodies produced by genetic recombination, are also included.

An intact protein or its partial peptide may be used as the antigen for immunization. As partial peptides of the proteins, for example, the amino (N)-terminal fragment of the protein and the carboxy (C)-terminal fragment can be given.

A gene encoding a protein of interest or a fragment thereof (e.g., an immunological fragment) is inserted into a known expression vector, and, by transforming the host cells with the vector described herein, the desired protein or a fragment thereof is recovered from outside or inside the host cells using standard methods. This protein can be used as the sensitizing antigen. Also, cells expressing the protein, cell lysates, or a chemically synthesized protein of the disclosures may be also used as a sensitizing antigen.

The mammal that is immunized by the sensitizing antigen is not restricted; however, it is preferable to select animals by considering the compatibility with the parent cells used in cell fusion. Generally, animals belonging to the orders rodentia, lagomorpha, or primates are used. Examples of animals belonging to the order of rodentia that may be used include, for example, mice, rats, and hamsters. Examples of animals belonging to the order of lagomorpha that may be used include, for example, rabbits. Examples of animals belonging to the order of primates that may be used include, for example, monkeys. Examples of monkeys to be used include the infraorder catarrhini (old world monkeys), for example, Macaca fascicularis, rhesus monkeys, sacred baboons, and chimpanzees.

Well-known methods may be used to immunize animals with the sensitizing antigen. For example, the sensitizing antigen is injected intraperitoneally or subcutaneously into mammals. Specifically, the sensitizing antigen is suitably diluted and suspended in physiological saline, phosphate-buffered saline (PBS), and so on, and mixed with a suitable amount of general adjuvant if desired, for example, with Freund's complete adjuvant. Then, the solution is emulsified and injected into the mammal. Thereafter, the sensitizing antigen suitably mixed with Freund's incomplete adjuvant is preferably given several times every 4 to 21 days. A suitable carrier can also be used when immunizing and animal with the sensitizing antigen. After the immunization, the elevation in the level of serum antibody is detected by usual methods.

Polyclonal antibodies against the proteins of the present disclosure can be prepared as follows. After verifying that the desired serum antibody level has been reached, blood is withdrawn from the mammal sensitized with antigen. Serum is isolated from this blood using conventional methods. The serum containing the polyclonal antibody may be used as the polyclonal antibody, or according to needs, the polyclonal antibody-containing fraction may be further isolated from the serum. For example, a fraction of antibodies that specifically recognize the protein of the invention may be prepared by using an affinity column to which the protein is coupled. Then, the fraction may be further purified by using a Protein A or Protein G column in order to prepare immunoglobulin G or M.

To obtain monoclonal antibodies, after verifying that the desired serum antibody level has been reached in the mammal sensitized with the above-described antigen, immunocytes are taken from the mammal and used for cell fusion. For this purpose, splenocytes can be mentioned as preferable immunocytes. As parent cells fused with the above immunocytes, mammalian myeloma cells are preferably used. More preferably, myeloma cells that have acquired the feature, which can be used to distinguish fusion cells by agents, are used as the parent cell.

The cell fusion between the above immunocytes and myeloma cells can be conducted according to known methods, for example, the method by Milstein et al. (Galfre et al., Methods Enzymol. 73:3-46, 1981).

The hybridoma obtained from cell fusion is selected by culturing the cells in a standard selection medium, for example, HAT culture medium (medium containing hypoxanthine, aminopterin, and thymidine). The culture in this HAT medium is continued for a period sufficient enough for cells (non-fusion cells) other than the objective hybridoma to perish, usually from a few days to a few weeks. Then, the usual limiting dilution method is carried out, and the hybridoma producing the objective antibody is screened and cloned.

Other than the above method for obtaining hybridomas, by immunizing an animal other than humans with the antigen, a hybridoma producing the objective human antibodies having the activity to bind to proteins can be obtained by the method of sensitizing human lymphocytes, for example, human lymphocytes infected with the EB virus, with proteins, protein-expressing cells, or lysates thereof in vitro and fusing the sensitized lymphocytes with myeloma cells derived from human, for example, U266, having a permanent cell division ability.

The monoclonal antibodies obtained by transplanting the obtained hybridomas into the abdominal cavity of a mouse and extracting ascites can be purified by, for example, ammonium sulfate precipitation, protein A or protein G column, DEAE ion exchange chromatography, an affinity column to which the protein of the present disclosure is coupled, and so on.

Monoclonal antibodies can be also obtained as recombinant antibodies produced by using the genetic engineering technique (see, for example, Borrebaeck C. A. K. and Larrick, J. W., THERAPEUTIC MONOCLONAL ANTIBODIES, Published in the United Kingdom by MACMILLAN PUBLISHERS LTD (1990)). Recombinant antibodies are produced by cloning the encoding DNA from immunocytes, such as hybridoma or antibody-producing sensitized lymphocytes, incorporating into a suitable vector, and introducing this vector into a host to produce the antibody. The present disclosure encompasses such recombinant antibodies as well.

Antibodies or antibody fragments specific for a protein encoded by one or more biomarkers can also be generated by in vitro methods such as phage display.

Moreover, the antibody of the present disclosure may be an antibody fragment or modified-antibody, so long as it binds to a protein encoded by a biomarker of the invention. For instance, Fab, F (ab′) 2, Fv, or single chain Fv (scFv) in which the H chain Fv and the L chain Fv are suitably linked by a linker (Huston et al., Proc. Natl. Acad. Sci. USA, 85:5879-5883, (1988)) can be given as antibody fragments. Specifically, antibody fragments are generated by treating antibodies with enzymes, for example, papain or pepsin. Alternatively, they may be generated by constructing a gene encoding an antibody fragment, introducing this into an expression vector, and expressing this vector in suitable host cells (see, for example, Co et al., J. Immunol., 152:2968-2976, 1994; Better et al., Methods Enzymol., 178:476-496, 1989; Pluckthun et al., Methods Enzymol., 178:497-515, 1989; Lamoyi, Methods Enzymol., 121:652-663, 1986; Rousseaux et al., Methods Enzymol., 121:663-669, 1986; Bird et al., Trends Biotechnol., 9:132-137, 1991).

The antibodies may be conjugated to various molecules, such as fluorescent substances, radioactive substances, and luminescent substances. Methods to attach such moieties to an antibody are already established and conventional in the field (see, e.g., U.S. Pat. Nos. 5,057,313 and 5,156,840).

Examples of methods that assay the antigen-binding activity of the antibodies include, for example, measurement of absorbance, enzyme-linked immunosorbent assay (ELISA), enzyme immunoassay (EIA), radioimmunoassay (RIA), and/or immunofluorescence. For example, when using ELISA, a protein encoded by a biomarker of the invention is added to a plate coated with the antibodies of the present disclosure, and then, the antibody sample, for example, culture supernatants of antibody-producing cells, or purified antibodies are added. Then, secondary antibody recognizing the primary antibody, which is labeled by alkaline phosphatase and such enzymes, is added, the plate is incubated and washed, and the absorbance is measured to evaluate the antigen-binding activity after adding an enzyme substrate such as p-nitrophenyl phosphate. As the protein, a protein fragment, for example, a fragment comprising a C-terminus, or a fragment comprising an N-terminus may be used. To evaluate the activity of the antibody of the invention, BIAcore (Pharmacia) may be used.

By using these methods, the antibody and a sample presumed to contain a protein of the disclosure are contacted, and the protein encoded by a biomarker of the disclosure is detected or assayed by detecting or assaying the immune complex formed between the above-mentioned antibody and the protein.

Mass spectrometry based quantitation assay methods, for example, but not limited to, multiple reaction monitoring (MRM)-based approaches in combination with stable-isotope labeled internal standards, are an alternative to immunoassays for quantitative measurement of proteins. These approaches do not require the use of antibodies and so the analysis can be performed in a cost- and time-efficient manner (see, for example, Addona et al., Nat. Biotechnol., 27:633-641, 2009; Kuzyk et al., Mol. Cell Proteomics, 8:1860-1877, 2009; Paulovich et al., Proteomics Clin. Appl., 2:1386-1402, 2008). In addition, MRM offers superior multiplexing capabilities, allowing for the simultaneous quantification of numerous proteins in parallel. The basic theory of these methods has been well-established and widely utilized for drug metabolism and pharmacokinetics analysis of small molecules.

In another embodiment, the expression level of a NOS2/iNOS of interest is determined by measuring RNA levels. A variety of suitable methods can be employed to detect and/or measure the level of mRNA expression of a gene. For example, mRNA expression can be determined using Northern blot or dot blot analysis, reverse transcriptase-PCR (RT-PCR; e.g., quantitative RT-PCR), in situ hybridization (e.g., quantitative in situ hybridization) or nucleic acid array (e.g., oligonucleotide arrays or gene chips) analysis. Details of such methods are described below and in, e.g., Sambrook et al., Molecular Cloning: A Laboratory Manual Second Edition vol. 1, 2 and 3. Cold Spring Harbor Laboratory Press: Cold Spring Harbor, N.Y., USA, November 1989; Gibson et al. (1999) Genome Res., 6(10):995-1001; and Zhang et al. (2005) Environ. Sci. Technol., 39(8):2777-2785; U.S. Publication No. 2004086915; European Patent No. 0543942; and U.S. Pat. No. 7,101,663; the disclosures of each of which are incorporated herein by reference in their entirety.

In one example, the presence or amount of one or more discrete mRNA populations in a surface skin sample can be determined by isolating total mRNA from the surface skin sample (see, e.g., Sambrook et al. (supra) and U.S. Pat. No. 6,812,341) and subjecting the isolated mRNA to agarose gel electrophoresis to separate the mRNA by size. The size-separated mRNAs are then transferred (e.g., by diffusion) to a solid support such as a nitrocellulose membrane. The presence or amount of one or more mRNA populations in the surface skin sample can then be determined using one or more detectably-labeled-polynucleotide probes, complementary to the mRNA sequence of interest, which bind to and thus render detectable their corresponding mRNA populations. Detectable-labels include, e.g., fluorescent (e.g., umbelliferone, fluorescein, fluorescein isothiocyanate, rhodamine, dichlorotriazinylamine fluorescein, dansyl chloride, allophycocyanin (APC), or phycoerythrin), luminescent (e.g., europium, terbium, Qdot™ nanoparticles supplied by the Quantum Dot Corporation, Palo Alto, Calif.), radiological (e.g., 1251, 1311, 35S, 32P, 33P, or 3H), and enzymatic (horseradish peroxidase, alkaline phosphatase, beta-galactosidase, or acetylcholinesterase) labels.

In another example, the presence or amount of discrete populations of mRNA (e.g., mRNA encoded by the NOS2/iNOS gene) in a surface skin sample can be determined using nucleic acid (or oligonucleotide) arrays. For example, isolated mRNA from a surface skin sample can be amplified using RT-PCR with, e.g., random hexamer or oligo(dT)-primer mediated first strand synthesis. The amplicons can be fragmented into shorter segments. The RT-PCR step can be used to detectably-label the amplicons, or, optionally, the amplicons can be detectably-labeled subsequent to the RT-PCR step. For example, the detectable-label can be enzymatically (e.g., by nick-translation or kinase such as T4 polynucleotide kinase) or chemically conjugated to the amplicons using any of a variety of suitable techniques (see, e.g., Sambrook et al., supra). The detectably-labeled-amplicons are then contacted with a plurality of polynucleotide probe sets, each set containing one or more of a polynucleotide (e.g., an oligonucleotide) probe specific for (and capable of binding to) a corresponding amplicon, and where the plurality contains many probe sets each corresponding to a different amplicon. Generally, the probe sets are bound to a solid support and the position of each probe set is predetermined on the solid support. The binding of a detectably-labeled amplicon to a corresponding probe of a probe set indicates the presence or amount of a target mRNA in the surface skin sample. Additional methods for detecting mRNA expression using nucleic acid arrays are described in, e.g., U.S. Pat. Nos. 5,445,934; 6,027,880; 6,057,100; 6,156,501; 6,261,776; and 6,576,424; the disclosures of each of which are incorporated herein by reference in their entirety.

Methods of detecting and/or for quantifying a detectable label depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Methods for detecting or measuring gene expression (e.g., protein or mRNA expression) can optionally be performed in formats that allow for rapid preparation, processing, and analysis of multiple samples. This can be, for example, in multi-welled assay plates (e.g., 96 wells or 386 wells) or arrays (e.g., nucleic acid chips or protein chips). Stock solutions for various reagents can be provided manually or robotically, and subsequent sample preparation (e.g., RT-PCR, labeling, or cell fixation), pipetting, diluting, mixing, distribution, washing, incubating (e.g., hybridization), sample readout, data collection (optical data) and/or analysis (computer aided image analysis) can be done robotically using commercially available analysis software, robotics, and detection instrumentation capable of detecting the signal generated from the assay. Examples of such detectors include, but are not limited to, spectrophotometers, luminometers, fluorimeters, and devices that measure radioisotope decay. Exemplary high-throughput cell-based assays (e.g., detecting the presence or level of a target protein in a cell) can utilize ArrayScan® VTI HCS Reader or KineticScan® HCS Reader technology (Cellomics Inc., Pittsburgh, Pa.).

In some embodiments, the expression level of the NOS2/iNOS gene and/or other gene biomarkers of this disclosure can be assessed and/or measured. To aid in detecting the presence or level of expression of the biomarker genes of interest, any part of the nucleic acid sequence of the genes can be used, e.g., as hybridization polynucleotide probes or primers (e.g., for amplification or reverse transcription). The probes and primers can be oligonucleotides of sufficient length to provide specific hybridization to an RNA, DNA, cDNA, or fragments thereof isolated from a surface skin sample. Depending on the specific application, varying hybridization conditions can be employed to achieve varying degrees of selectivity of a probe or primer towards target sequence. The primers and probes can be detectably-labeled with reagents that facilitate detection (e.g., fluorescent labels, chemical labels (see, e.g., U.S. Pat. Nos. 4,582,789 and 4,563,417), or modified bases).

Standard stringency conditions are described by Sambrook, et al. (supra) and Haymes, et al. Nucleic Acid Hybridization, A Practical Approach, IRL Press, Washington, D.C. (1985). In order for a nucleic acid molecule to serve as a primer or probe it need only be sufficiently complementary in sequence to be able to form a stable double-stranded structure under the particular hybridization conditions (e.g., solvent and salt concentrations) employed.

Hybridization can be used to assess homology between two nucleic acid sequences. A nucleic acid sequence described herein, or a fragment thereof, can be used as a hybridization probe according to standard hybridization techniques. The hybridization of a probe of interest (e.g., a probe containing a portion of a nucleotide sequence described herein or its complement) to DNA, RNA, cDNA, or fragments thereof from a test source is an indication of the presence of DNA or RNA corresponding to the probe in the test source. Hybridization conditions are known to those skilled in the art and can be found in Current Protocols in Molecular Biology, John Wiley & Sons, N.Y., 6.3.1-6.3.6, 1991. Moderate hybridization conditions are defined as hybridization in 2× sodium chloride/sodium citrate (SSC) at 30° C., followed by a wash in 1×SSC, 0.1% SDS at 50° C. Highly stringent conditions are defined as hybridization in 6×SSC at 45° C., followed by a wash in 0.2×SSC, 0.1% SDS at 65° C.

Primers can be used in in a variety of PCR-type methods. For example, polymerase chain reaction (PCR) techniques can be used to amplify specific sequences from DNA as well as RNA, including sequences from total genomic DNA or total cellular RNA. The PCR primers are designed to flank the region that one is interested in amplifying. Primers can be located near the 5′ end, the 3′ end or anywhere within the nucleotide sequence that is to be amplified. The amplicon length is dictated by the experimental goals. For qPCR, the target length is closer to 100 base pairs and for standard PCR, it is near 500 base pairs. Generally, sequence information from the ends of the region of interest or beyond is employed to design oligonucleotide primers that are identical or similar in sequence to opposite strands of the template to be amplified. PCR primers can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair.

In addition, the nucleic acid sequences or fragments thereof (e.g., oligonucleotide probes) can be used in nucleic acid arrays for detection and/or quantitation of gene expression.

### 10. Methods of Treatment

The methods disclosed herein enable the diagnosis of psoriasis based on biomarker levels in a subject's surface skin, followed by treatment of said subject with an appropriate psoriasis therapy. The methods disclosed herein enable the assessment whether or not a subject having or suspected of having psoriasis is likely to respond to a psoriasis therapy. A subject having or suspected of having psoriasis who is likely to respond to the psoriasis therapy can be administered the psoriasis therapy. Conversely, a subject having or suspected of having psoriasis who is not likely to respond to a psoriasis therapy can be administered a different psoriasis therapy that is suitable for treatment of psoriasis.

The methods of this disclosure also enable the stratification of subjects having or suspected of having psoriasis into groups of subjects that are more likely to benefit, and groups of subjects that are less likely to benefit, from treatment comprising a psoriasis therapy. The ability to select such subjects from a pool of psoriasis subjects who are being considered for treatment with a psoriasis therapy is beneficial for administering an effective treatment to the subject.

The subjects who are considered for treatment comprising a psoriasis therapy include, but are not limited to, subjects having, suspected of having, or likely to develop psoriasis. In one embodiment, the subject to be treated with a psoriasis therapy has, is suspected of having, or is likely to develop mild psoriasis. In one embodiment, the subject to be treated with a psoriasis therapy has, is suspected of having, or is likely to develop moderate psoriasis. In one embodiment, the subject to be treated with a psoriasis therapy has, is suspected of having, or is likely to develop severe psoriasis.

If the subject having psoriasis is more likely to respond to a psoriasis therapy (based on levels of NOS2/iNOS biomarker alone or in combination with any of the other biomarkers of this disclosure described elsewhere in this disclosure), the subject can then be administered an effective amount of the psoriasis therapy. An effective amount of the compound can suitably be determined by a health care practitioner taking into account, for example, the characteristics of the patient (age, sex, weight, race, etc.), the progression of the disease, and prior exposure to the drug. If the subject is less likely to respond to one psoriasis therapy, the subject can then be optionally administered a different psoriasis therapy.

In addition to the prediction of the likelihood of treatment effectiveness in a patient with psoriasis, the progress of a psoriasis treatment can be followed by monitoring the levels of the biomarkers described above. In some embodiments, a method of assessing or monitoring the effectiveness of a psoriasis treatment in a subject is provided. A surface skin sample is obtained from the subject, and the levels of one or more of the above-described biomarkers, e.g., NOS2/iNOS biomarker, are measured to determine whether the biomarker levels are increased or decreased compared to the levels prior to the initiation of the treatment.

In one embodiment, provided herein is a method of monitoring subject response to a psoriasis treatment comprising: obtaining a surface skin sample from the subject; measuring the level of NOS2/iNOS biomarker and optionally, one or more markers selected from IL-17C, IL-1β, IL-6, CXCL8/IL-8, TNFa, IFNγ, CXCL9, IL-17A, IL-17F, IL-23p19, IL-12/IL-23p40, IL-36A, IL-36G, DEFB4B, CCL20, CXCL1, CAMP/LL37, PI3, STAT3, S100A9, and S100A12, in the first sample; administering a psoriasis treatment compound to the subject; thereafter obtaining a second surface skin sample from the subject; measuring the level of NOS2/iNOS biomarker and optionally, one or more markers selected from IL-17C, IL-1β, IL-6, CXCL8/IL-8, TNFa, IFNγ, CXCL9, IL-17A, IL-17F, IL-23p19, IL-12/IL-23p40, IL-36A, IL-36G, DEFB4B, CCL20, CXCL1, CAMP/LL37, PI3, STAT3, S100A9, and S100A12, in the second sample; and comparing the levels of the biomarkers obtained from first and second samples; wherein a decreased level of the biomarkers in the second sample indicates an effective response.

The biomarkers can also be used to track and adjust individual patient treatment effectiveness. The biomarkers can be used to gather information needed to make adjustments in a patient's treatment, increasing or decreasing the dose of an agent as needed. For example, a patient receiving a treatment compound can be tested using a biomarker to see if the dosage is becoming effective, or if a more aggressive treatment plan may be needed.

Subjects of all ages can be affected by psoriasis. Therefore, a surface skin sample used in a method described herein can be obtained from a human subject of any age, including a fetus, an infant, a child, an adolescent, or an adult, such as an adult having, or suspected of having, psoriasis.

The methods can also be applied to individuals at risk of developing psoriasis treatable by a psoriasis therapy. Such individuals include those who have (i) a family history of (a genetic predisposition for) such disorders or (ii) one or more risk factors for developing such disorders.

After stratifying or selecting a subject based on whether the subject will be more likely or less likely to respond to a psoriasis therapy, a medical practitioner (e.g., a doctor) can administer the appropriate therapeutic modality to the subject. Methods of administering psoriasis therapies are known in the art.

It is understood that any therapy described herein can include one or more additional therapeutic agents. That is, any therapy described herein can be co-administered (administered in combination) with one or more additional therapeutic agents such as, but not limited to, other psoriasis therapies described herein. Furthermore, any therapy described herein can include one or more agents for treating, or more side-effects of a therapy comprising the psoriasis therapy. Combination therapies (e.g., co-administration of a psoriasis therapy and one or more additional psoriasis therapies or additional therapeutic agents) can be, e.g., simultaneous or successive. For example, a psoriasis therapy and the additional therapeutic agent(s) can be administered at the same time or at different times. In some embodiments, the one or more additional therapeutic agents can be administered first in time and the psoriasis therapy can be administered second in time.

In cases where the subject having psoriasis and predicted to respond to a psoriasis therapy has been previously administered the psoriasis therapy, the therapy can replace or augment a previously or currently administered therapy. For example, upon treating with corticosteroid, administration of a non-corticosteroid therapy can cease or diminish, e.g., be administered at lower levels. Administration of the previous therapy can be maintained while the therapy comprising corticosteroid is administered. In some embodiments, a previous therapy can be maintained until the level of corticosteroid reaches a level sufficient to provide a therapeutic effect.

### 11. Kits

This disclosure also provides kits. In certain embodiments, the kit can include an antibody or antibodies that can be used to detect one or more of the biomarkers disclosed herein or their concentration or expression levels. For example, the kit can include an antibody that specifically binds NOS2/iNOS. The antibodies in the kit may be monoclonal or polyclonal and can be further conjugated with a detectable label. In some embodiments, the kit includes probes that can be used to identify or detect any of the biomarkers disclosed herein. In some embodiments, the kit includes any of the nucleic acid arrays. In some embodiments, the kit includes probes and antibodies that can be used to identify or detect any of the biomarkers disclosed herein or their expression or expression levels. The kits can, optionally, contain instructions for detecting and/or measuring the concentration of one or more proteins or the levels of mRNA in a surface skin sample.

The kits can optionally include, e.g., a control (e.g., a concentration standard for the protein being assessed) or control labeled-amplicon set containing known amounts of one or more amplicons recognized by nucleic acid probes of the array. In some instances, the control can be an insert (e.g., a paper insert or electronic medium such as a CD, DVD, or floppy disk) containing an expression level or expression level ranges of one or more proteins (e.g., NOS2/iNOS) or RNAs predictive of psoriasis, or of responsiveness to a psoriasis therapy.

In some embodiments, the kits can include one or more reagents for processing a surface skin sample (e.g., calibration reagents, buffers, diluents, color reagents, reagents to stop a reaction). For example, a kit can include reagents for isolating a protein from a surface skin sample and/or reagents for detecting the presence and/or amount of a protein in a surface skin sample (e.g., an antibody that binds to the protein that is the subject of the detection assay and/or an antibody that binds the antibody that binds to the protein).

In certain embodiments, the kit includes at least one microplate (e.g., a 96 well plate; i.e., 12 strips of 8 wells). The microplate can be provided with its corresponding plate cover. The microplate can be polystyrene or of any other suitable material. The microplate can have the antibody that is used to identify the presence of a particular biomarker coated inside each well. The antibody may be conjugated to a detectable label. The kit may also include at least one adhesive strip.

In some embodiments, the kits can include a software package for analyzing the results of, e.g., expression profile or a microarray analysis.

The kits can also include one or more antibodies for detecting the protein expression of any of the genes described herein (e.g., NOS2/iNOS). For example, a kit can include (or in some cases consist of) one or a plurality of antibodies capable of specifically binding to one or more proteins encoded by any of the genes described herein and optionally, instructions for detecting and/or measuring the concentration of one or more proteins and/or a detection antibody comprising a detectably-labeled antibody that is capable of binding to at least one antibody of the plurality. In some embodiments, the kits can include antibodies that recognize one or more of NOS2/iNOS, IL-17C, IL-1β, IL-6, CXCL8/IL-8, TNFa, IFNγ, CXCL9, CXCL10, IL-17A, IL-17F, IL-23p19, IL-12/IL-23p40, IL-36A, IL-36G, DEFB4B, CCL20, CXCL1, CAMP/LL37, PI3, STAT3, S100A9, and S100A12, and SERPINB3. In some embodiments, the kits can include antibodies that recognize NOS2/iNOS. In certain embodiments, the kit can also optionally include one or more unit doses of psoriasis therapy.

The kits described herein can also, optionally, include instructions for administering a psoriasis therapy, where the concentration of one or more proteins or expression level of one or more RNAs predicts that a subject having or suspected of having psoriasis will respond to a psoriasis therapy.

In a specific embodiment, the kit comprises one or more of the following:

(i) a microplate (e.g., a 96 well plate). The microplate can be coated with an anti-NOS2/iNOS antibody that is conjugated with a detectable label. The anti-NOS2/iNOS antibody may monoclonal or polyclonal. The antibody can be e.g., from mouse, rabbit, rat, or guinea pig. The detectable label can be e.g., horse radish peroxidase, biotin, a fluorescent moiety, a radioactive moiety, a histidine tag, or a peptide tag. The microplate can be provided with a cover and optionally, one or more adhesive strips.

(ii) a vial containing anti-NOS2/iNOS conjugated with a detectable label. The detectable label can be e.g., horse radish peroxidase, biotin, a fluorescent moiety, a histidine tag, a peptide tag. The vial can also include a preservative.

(iii) a vial containing an NOS2/iNOS standard of known concentration. The NOS2/iNOS can be a recombinant human NOS2/iNOS.

(iv) a vial containing an assay diluent.

(v) a vial containing a calibrator diluent.

(vi) a vial containing wash buffer. The buffer may be provided as a concentrate.

(vii) one or more vials containing color reagents.

(viii) a vial containing a stop solution to stop the colorimetric reaction.

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### EXAMPLES

### Example 1: Tape-Strips to Detect Distinct Immune and Barrier Profiles in Atopic Dermatitis and Psoriasis

A global transcriptome of tape-strips from lesional/non-lesional skin of adults with moderate to severe atopic dermatitis (AD) and psoriasis was constructed using the following methods.

### Study Population and Characteristics

20 adults with moderate-to-severe AD (15 female, 5 male, mean age 31) and psoriasis (8 female, 12 male, mean age 42.6), and 20 healthy volunteers (11 female, 9 male, mean age 40.3) were enrolled, under institutional review board-approved protocols. Clinical severity scores including a 5-grade AD Investigator Global Assessment (IGA; mean 2.6) and Total Sign Score (TSS; mean 7.6), Physician Global Assessment (PGA; mean 3.1) and Target Lesion Severity Score (TLSS; mean 8.9), were measured for AD and psoriasis, respectively, prior to tape-stripping, as shown in Table 1). Exclusion criteria included immunodeficiency, use of biologics within 12 weeks, systemic steroids, immunosuppressants, or phototherapy within 4 weeks, and topical medication within 2 weeks.

TABLE 1
Clinical demographics

| | | Atopic | | |
|---|---|---|---|---|
| | | dermatitis | Psoriasis | Controls |
| Characteristic | | (n = 20) | (n = 20) | (n = 20) |
| Age (years) | | 31.0 ± 13.8 | 42.6 ± 15.4 | 40.3 ± 15.7 |
| mean ± SD | | | | |
| Gender | | | | |
| Female | | 15 | 8 | 11 |
| Male | | 5 | 12 | 9 |
| Race | | | | |
| Caucasian | | 18 | 19 | 20 |
| African American | | 2 | 0 | 0 |
| Asian | | 0 | 1 | 0 |
| Clinical Severity | | | | |
| TSS, mean ± SD (range) | | 7.6 ± 2.5 | — | — |
| | | (4-15) | | |
| IGA, mean ± SD | | 2.6 ± 0.6 | — | — |
| Number of patients with IGA | | | | |
| score | IGA 2 (mild) | 8 | — | — |
| | IGA 3 (moderate) | 11 | — | — |
| | IGA 4 (severe) | 1 | — | — |
| TLSS, mean ± SD (range) | | — | 8.9 ± 1.8 | — |
| | | | (5-12) | |
| PGA, mean ± SD | | — | 3.1 ± 0.6 | — |
| Number of patients with PGA | | | | |
| score | PGA 2 (mild) | — | 3 | — |
| | PGA 3 (moderate) | — | 12 | — |
| | PGA 4 (severe) | — | 5 | — |

AD: Atopic Dermatitis;
SD: Standard Deviation;
TSS: Total Sign Score;
IGA: Investigator’s Global Assessment;
TLSS: Target Lesion Severity Score;
PGA: Physician’s Global Assessment

Tape-Strip Collection, RNA Extraction, qRT-PCR and RNA-Sequencing

Twenty consecutive large D-Squame tape-strips (CuDerm, Dallas, Tex.) were collected from representative lesions and non-lesional skin (in close proximity, but >10 cm away from lesions) in the extremities of all AD and psoriasis patients, and skin from controls. Each large D-Squame (D102) tape-strip was applied for a few seconds to the antecubital fossa when possible for lesional skin. Using a pen, small marks were made on the skin after placement of the first tape-strip to ensure consistent placement of each subsequent tape-strip. Thus, all 20 tapes for lesional or non-lesional skin were placed on exactly the same skin area. After cleansing with an alcohol pad, tape-strips were placed on the skin and pressure was applied manually with fingers for approximately 5 seconds. Tapes were removed, placed on a D-Squame cardboard, and immediately frozen on dry ice. The cardboards were subsequently kept in an −80° C. freezer.

The tapes were pooled together and thoroughly washed with QIAzol for RNA extraction using miRNAeasy Mini Kit. (Qiagen, Hilden, Germany). The mean RNA yield across tape-strips samples was 39.7±56.6 ng. For qRT-PCR, 500 pg of RNA per sample were subjected to reverse transcription, pre-amplification, and qRT-PCR, as previously described (Sanyal R D et al., Ann Allergy Asthma Immunol 2019; 122:99-110). Primers are listed in Table 2. RNA AmpliSeq libraries were constructed with the Ion AmpliSeq Transcriptome Human Gene Expression Kit using 5 ng of RNA per sample and an amplification approach that screens>20,000 genes per reaction. RNA-seq libraries were pooled and sequenced on the Ion S5 XL system sequencer with Ion 550 Chips.

TABLE 2
Immune and barrier primers used for qRT-PCR

| Gene | Assay ID |
|---|---|
| CAMP/LL37 | Hs00189038_m1 |
| CCL2 | Hs00234140_m1 |
| CCL5 | Hs00174575_m1 |
| CCL7 | Hs00171147_m1 |
| CCL13 | Hs00234646_m1 |
| CCL17 | Hs00171074_m1 |
| CCL18 | Hs00268113_m1 |
| CCL19 | Hs00171149_m1 |
| CCL20 | Hs00355476_m1 |
| CCL22 | Hs01574247_m1 |
| CCL26 | Hs00171146_m1 |
| CCL27 | Hs04399123_g1 |
| CCR4 | Hs99999919_m1 |
| CCR6 | Hs01890706_s1 |
| CCR7 | Hs01013469_m1 |
| CCR10 | Hs00706455_s1 |
| CD3 | Hs00174158_m1 |
| CD207/Langerin | Hs00210453_m1 |
| CD209/DC-SIGN | Hs01588349_m1 |
| CDSN | Hs00169911_m1 |
| CLA/CD162 | Hs05033974_s1 |
| CLDN8 | Hs04186769_s1 |
| CLDN23 | Hs01013638_s1 |
| CSF1/M-CSF | Hs00174164_m1 |
| CXCL1 | Hs00236937_m1 |
| CXCL2 | Hs00601975_m1 |
| CXCL9 | Hs00171065_m1 |
| CXCL10 | Hs01124252_g1 |
| DEFB4B | Hs00823638_m1 |
| ELOVL3 | Hs00537016_m1 |
| EREG | Hs00914313_m1 |
| FA2H | Hs00757813_m1 |
| FceR1A | Hs00175408_m1 |
| FLG | Hs00856927_g1 |
| FLG2 | Hs00418578_m1 |
| FOXP3 | Hs01085834_m1 |
| GZMA | Hs00989184_m1 |
| IL-10 | Hs00961622_m1 |
| IL-12/IL-23p40 | Hs01011518_m1 |
| IL-13 | Hs00174379_m1 |
| IL-15 | Hs01003716_m1 |
| IL-17A | Hs00174383_m1 |
| IL-17C | Hs00171163_m1 |
| IL-17F | Hs00369400_m1 |
| IL-19 | Hs00604657_m1 |
| IL-20 | Hs00218888_m1 |
| IL-21 | Hs00222327_m1 |
| IL-22 | Hs01574154_m1 |
| IL-23p19 | Hs00900828_g1 |
| IL-26 | Hs00218189_m1 |
| IL-31 | Hs01098710_m1 |
| IL-33 | Hs04931857_m1 |
| IL-34 | Hs01050926_m1 |
| IL-36A | Hs00205367_ml |
| IL-36B | Hs00758166_m1 |
| IL-36G | Hs00219742_m1 |
| IL-36RN | Hs01104220_g1 |
| IL-37 | Hs00367201_m1 |
| ITGAM/CD11b | Hs00167304_m1 |
| ITGAX/CD11c | Hs00174217_m1 |
| Ki67 | Hs04260396_g1 |
| KLK5 | Hs01548153_m1 |
| KLK7 | Hs00192503_m1 |
| KRT16 | Hs00955082_g1 |
| LCE2B | Hs04194422_s1 |
| LOR | Hs01894962_s1 |
| MMP12 | Hs00159178_m1 |
| iNOS (NOS2) | Hs01075529_m1 |
| OSM | Hs00171165_m1 |
| OSMR | Hs00384276_m1 |
| OX40 (TNFRSF4) | Hs00937194_g1 |
| OX40L (TNFSF4) | Hs04400731_m1 |
| PDE4B | Hs00963643_m1 |
| PDL1 (CD274) | Hs00204257_m1 |

### Statistical Analysis

Statistical analyses were performed using the statistical language R. Sample quality was assessed using FastQC. Samples were aligned to the human reference genome, using STAR (open source aligner; Dobin A et al., Bioinformatics 2013; 29:15-21). Mapped sequencing reads were assigned to genomic features using the Feature Counts function. Counts were transformed to log-scale by voom transform (Law C W et al., Genome Biol 2014; 15:R29) and fit in a linear model. Fold-changes/FCHs were estimated, and hypothesis testing was conducted using contrasts under the general framework for linear models in the limma package. P-values were adjusted for multiple hypotheses using the Bejamini-Hochberg procedure, controlling for false discovery rate/FDR. Genes with FCH>2 and FDR<0.05 were considered differentially expressed genes/DEGs. qRT-PCR values were normalized to the housekeeping gene Rplp0. Values below the limit of detection were imputed as 20% of the minimal expression value. Since our primarily European American cohort included two African American AD patients and one Asian psoriasis patient, we performed a sensitivity analysis with only Caucasian patients, which did not alter the significance of our findings. Spearman correlation coefficients and one-way ANOVA were used to evaluate correlations between biomarkers and TSS/TLSS and IGA/PGA, respectively. To evaluate the performance of a molecular classifier that discriminates between AD and psoriasis, we used the receiver operating characteristic under the curve (ROC AUC).

### Results

Tape-strips were obtained from lesional and non-lesional skin of patients with AD, psoriasis, and skin from healthy volunteers (n=20 each), and profiled using an RNA-seq approach that screens>20,000 transcripts (Li W et al., BMC Genomics 2015; 16:1069) followed by qRT-PCR analysis of disease-related immune and barrier biomarkers. For RNA-seq, sample recovery rates were: 20/20 (100%) and 19/20 (95%) for lesional and non-lesional psoriasis, respectively; 19/20 (95%) and 19/20 (95%) for lesional and non-lesional AD, respectively; and 19/20 (95%) for controls, with overall 96% sample recovery rate (96 of 100 samples). mRNA was acquired from all 100 samples (100%) by qRT-PCR. Tape-stripped skin showed Th2-skewing in AD and Th17 in psoriasis. We used fold-change/FCH>2 and false discovery rate/FDR<0.05 to define differentially expressed genes (DEGs). For AD versus control comparisons, we identified 4,123 DEGs (2,563 up, 1,560 down) in lesional and 1,498 DEGs (1,176 up, 322 down) in non-lesional skin. For psoriasis versus control comparisons, 5,390 genes (1,578 up, 3,812 down) were differentially expressed in lesional skin, while 1,135 (915 up, 220 down) were differentially expressed in non-lesional skin (data not shown). When comparing lesional to normal skin, 1,752 DEGs were detected to be unique to AD (1,270 up, 482 down), 3,019 unique to psoriasis (285 up, 2,734 down), and 2,371 shared by both diseases (1,293 up, 1,078 down). For non-lesional versus healthy tissues, 1,007 DEGs were unique to AD (727 up, 280 down), 644 were unique to psoriasis (466 up, 178 down), and 491 were shared (449 up, 42 down). 4,549 DEGs were identified in lesional versus non-lesional psoriasis skin (371 up, 4,178 down). In contrast, most AD lesional versus non-lesional differences were not significant enough to pass the FDR threshold (data not shown).

A heatmap was constructed which depicted a set of immune-related genes, which were also used in prior studies (refs 2, 3, 47, 48 to be added) (data not shown). While the lesional tape-strips of both diseases displayed high levels of immune activation, an overall greater inflammatory tone characterized non-lesional AD skin compared to psoriasis (data not shown).

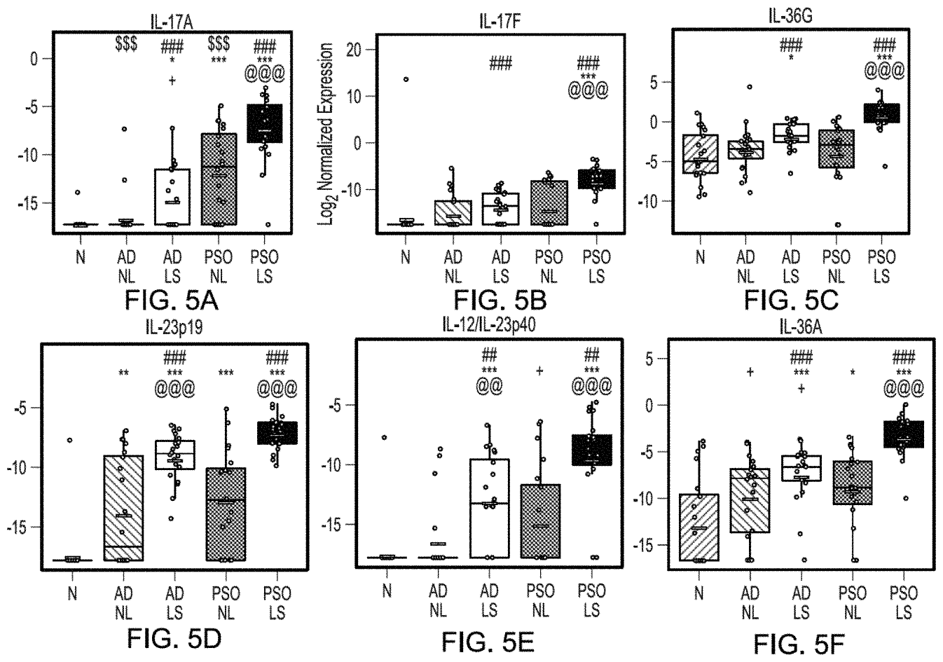

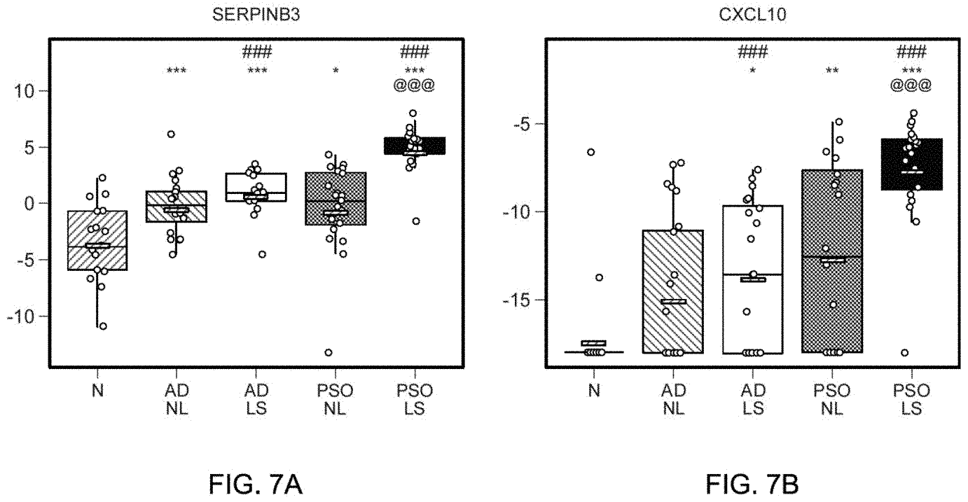

Several genes were commonly up-regulated in AD and psoriasis tissues versus controls, including markers of epidermal hyperplasia (SERPINB3/SERPINB4) (see FIG. 7A), general inflammation (MMP12), dendritic cells (DC)/macrophages (FCER1G, ITGAX/CD11c, CD83, CD86, LAMP3/DC-LAMP, CSF2), T-cell proliferation and migration (CD4, CD69, CD80, CCR7), and JAK/STAT signaling (JAK3, STAT4) (data not shown). ITGAM/CD11b, which is expressed on various leukocytes including DCs, macrophages, eosinophils, and neutrophils, as well as FCER1A, the high-affinity IgE component expressed on mast cells and inflammatory dendritic epidermal cells, were increased in both diseases, but showed preferential elevation in AD. ICOS, a marker of T-cell activation, and CD1A, which is expressed by DCs and Langerhans cells, were significantly increased only in AD, but not psoriasis. Negative regulators (IL-34, IL-37, IL-1F10) were commonly down-regulated in lesions of both diseases, similar to biopsy studies (all FDR<0.05).2, 3, 7, 49, 50 While AD and psoriasis shared increases in markers defining cellular infiltrates, distinct immune signatures separated the diseases. Psoriasis lesional and/or non-lesional tape-strips were most notable for prominent Th17-skewing, with significant increases in many Th17 products (IL-17A/F, IL-36A/G, CCL20, VNN3, DEFB4A/B, LCN2/lipocalin-2) that were either unchanged or showed more modest changes in AD (FIG. 5). IL-19, which is induced by both IL-17 and IL-4/IL-13, is the only IL-17-induced cytokine with higher expression in AD than psoriasis.

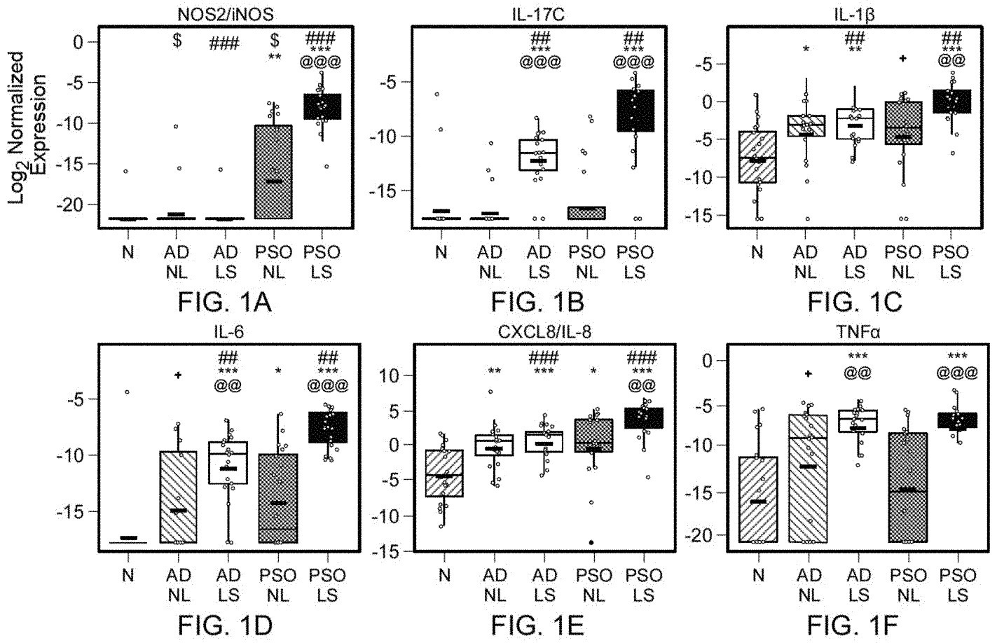

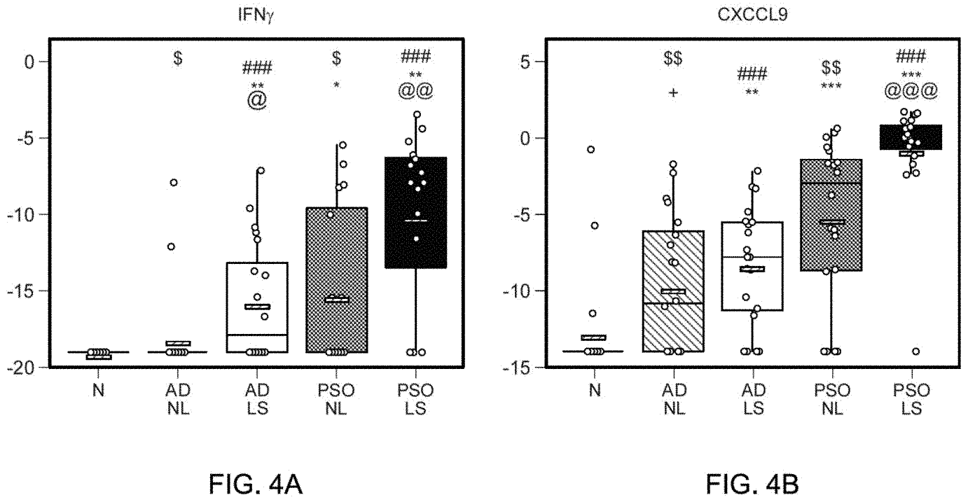

Psoriasis tissues also demonstrated preferential significant up-regulation of Th1 IFNG, CXCL9/CXCL10/CXCL11, CCL2/CCL3/CCL4, STAT1) and innate immunity (NOS2/iNOS, IL-1B, IL-17C) products, compared to both controls and AD (FIGS. 1, 3-5 and 7). Contrary, most Th2-related genes (IL-13, IL-10, IL-31, CCL13, CCL17/TARC, CCL22, CCL24/eotaxin-2, CCR4, TNFRSF4/OX40) were significantly increased in AD versus controls and/or psoriasis. Most of these Th2 products (e.g. IL-13, CCL17/TARC, CCL24/eotaxin-2, TNFRSF4/OX40) were significantly increased in both lesional and non-lesional AD, while the pruritus mediator, IL-31, was uniquely up-regulated in lesional AD. Th22-related (IL-22, S100As) genes shared up-regulation in both diseases (all FDR<0.05). When comparing lesional versus non-lesional tape-stripped skin, significant differences were not detected in AD, while in psoriasis, key biomarkers, including Th17/Th22- (IL36A, CCL20, VNN3, LCN2/lipocalin-2, S100As) and Th1-related (CXCL9/CXCL10) were significantly increased in lesional skin (FDR<0.05 (FIGS. 4-7).

### Distinct Barrier Alterations in AD and Psoriasis

A previously defined epidermal barrier gene-subset was also assessed in this study. Terminal differentiation (FLG2, ANXA9, LCE5A, SCEL), gap/tight junction (GJB3/GJB5, CLDN8), keratin (KRT77/KRT79), and lipid biosynthesis/metabolism (GAL, FABP7, FA2H, ALOXE3) products were significantly down-regulated in tape-strips from both AD and psoriasis lesions versus controls, while few barrier genes (ELOVL5, CLDN7, ANXA5/6) were increased in both diseases. Lipid biosynthesis/metabolism-related abnormalities were overall more pronounced in psoriasis, and several genes related to lipid processing were significantly down-regulated only in psoriasis (FADS2, FAR2, SPTLC1, GPAM). The late cornified envelope/LCE components (LCE3A/LCE3C) were preferentially up-regulated in psoriasis, as described in biopsy studies.12, 53-55 In contrast, terminal differentiation (PSORS1C2) and cadherin (CDH12) genes were significantly decreased only in AD (all FDR<0.05).

### Pathway Analyses Highlight Enrichment of Disease-Specific Axes

Gene-set variation analysis/GSVA was performed to evaluate enrichment of previously published immune and T-helper pathway gene-sets. While immune genes were significantly enriched in lesional and non-lesional tape-stripped skin of both diseases (P<0.01), the Th2 pathway was highest in AD (P<0.05), with lesser or no significant changes in psoriasis. Conversely, Th1/Th17 axes were highest in psoriasis, with lesser, but significant up-regulations in AD versus controls (P<0.05). The Th22/IL-22 axis showed similar up-regulation in both diseases (P<0.01). Down-regulation of lipid-related genes was greatest in psoriasis lesions, with significant down-regulation also seen in AD versus controls (P<0.05).

### Comparison of Tape-Strips to Biopsies

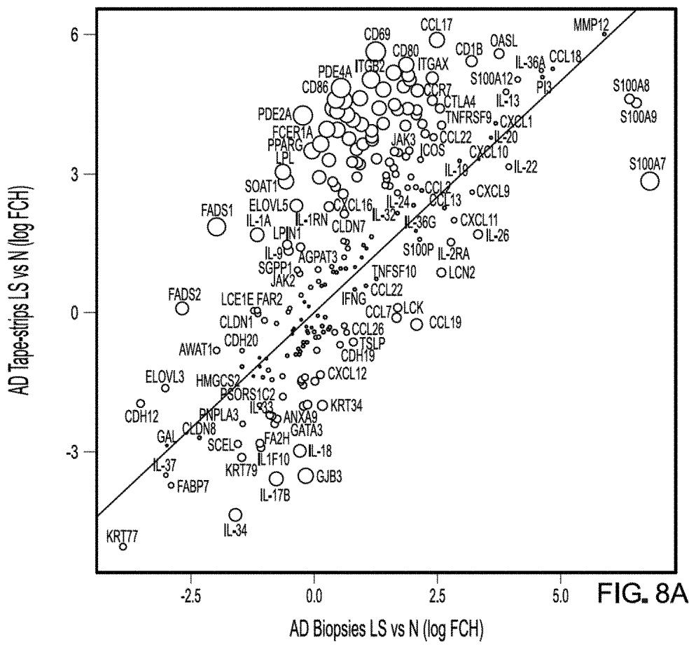

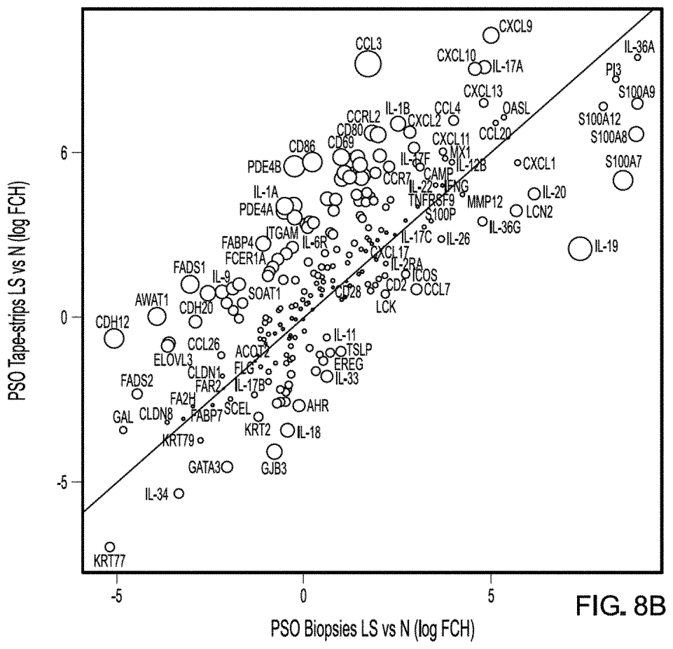

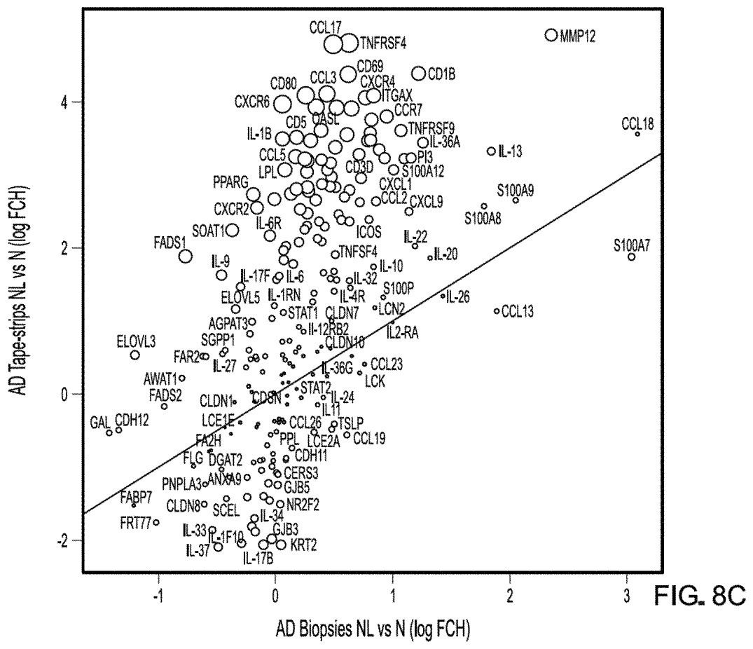

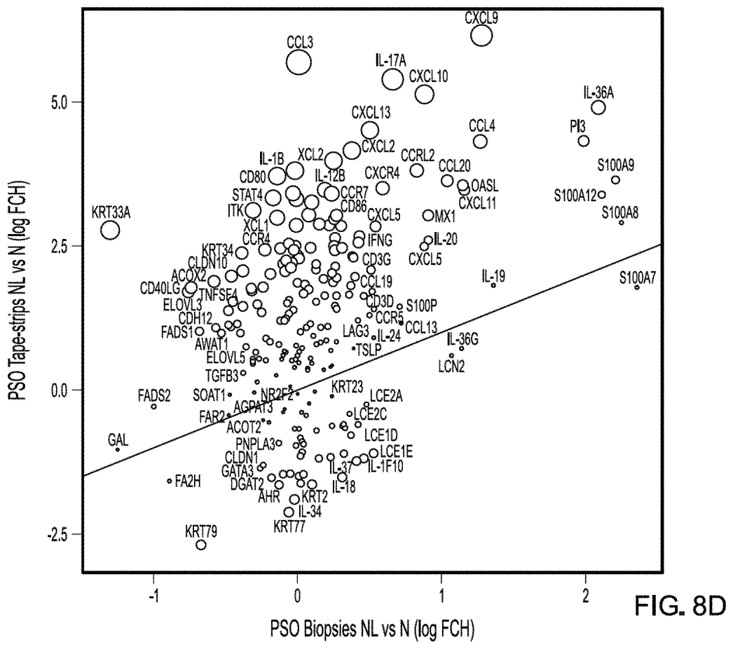

The tape-strip transcriptome of AD and psoriasis to biopsies from patients with similar disease severity was compared, using the cohort published by Tsoi, et al. J Invest Dermatol 2019; 139:1480-9. Overall, for both AD and psoriasis, diseased versus healthy skin differences were highly correlated between tape-strips and biopsies (FIG. 8). Taking the intersection of 14,313 genes that were evaluated in both studies, lesional AD demonstrated 3,958 DEGs in tape-strips and 1,307 DEGs in biopsies, while lesional psoriasis showed 5,208 DEGs in tape-strips and 2,706 DEGs in biopsies. In AD and psoriasis lesions respectively, 679 and 943 DEGs overlapped between tape-strips and biopsies. In both diseases, various DC/T-cell-related markers (FCER1A, ITGAX/CD11c, CD69, CD80, CD86), negative regulators (IL-34, IL-1F10), and barrier markers (GJB3, KRT79, PSORS1C2) displayed greater fold-changes in tape-strips, while the S100As and some lipid-related genes (FADS1/2, AWAT1) conversely showed greater differences in biopsies. Furthermore, AD lesions showed greater fold-changes for Th2 chemokine CCL17/TARC in tape-strips compared to biopsies. In psoriasis, tape-strips demonstrated greater fold-changes for Th1 chemokines (CXCL9/10, CCL3), although Th17 markers (IL-36G, IL-19, LCN2/lipocalin-2) were more dysregulated in biopsies. Tape-strips displayed markedly greater dysregulation in non-lesional skin compared to biopsies for both AD (1,450 DEGs in tapes, 81 DEGs in biopsies) and psoriasis (1,071 DEGs in tapes, 43 DEGs in biopsies). Tape-strips, but not biopsies, showed significant up-regulation of DC/T-cell markers (CD3D, CD69, CD80, CD86, CCR7) for both diseases, Th2 gene (CCL17/TARC, TNFRSF40/OX40) for AD, and Th1/Th17 genes (CXCL9/10, CCL3, IL-17A, IL-36A, PI3) for psoriasis. qRT-PCR confirms and expands RNA-seq data. To validate the RNA-seq data, and to assess key immune and barrier markers that are often below detection even in RNA-seq,3, 56 a large panel of 99 genes representing cellular, immune, and barrier markers by qRT-PCR was evaluated. 99 of 99 (100%) mRNA products in all samples (100% detection) were quantified.

Cellular markers defining immune cell-subsets, such as macrophages, T-cells, and DCs/LCs, were increased in both lesional AD and psoriasis versus controls. These markers were mostly also significantly elevated in non-lesional AD, but not in psoriasis (P<0.05). The T-cell activation marker, ICOS, and CCR4, which defines activated Th2 cells, and the DC marker, CD209/DC-SIGN, were significantly higher in AD versus psoriasis lesions (P<0.05).

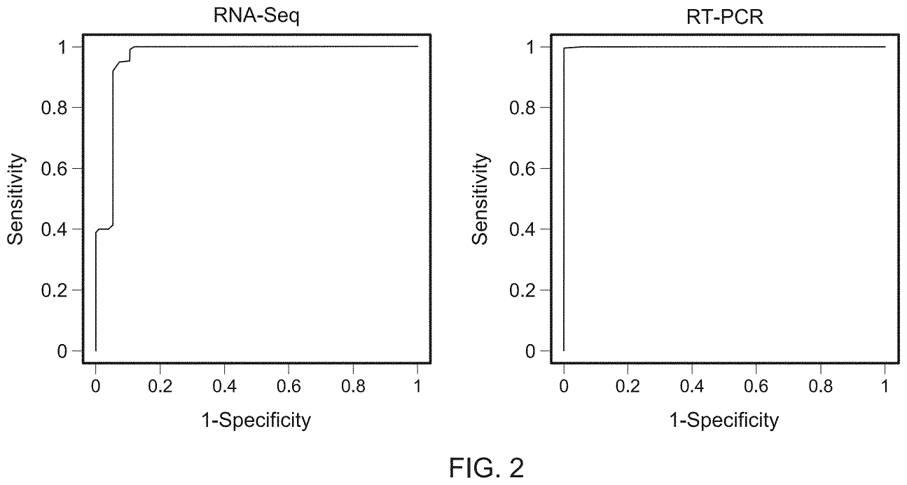

Innate immune markers (NOS2/iNOS, IL-17C, IL-1B, IL-6, CXCL8/IL-8) were significantly higher in psoriasis lesions versus both controls and AD lesions (P<0.01), with NOS2/iNOS, IL-6, and CXCL8/IL-8 also harboring increases in non-lesional psoriasis (P<0.05) (FIG. 1). Surprisingly, NOS2/iNOS levels were found to be 10,240 fold higher in tape-stripped psoriatic lesions versus controls while there was no change in AD lesions versus controls (FIG. 1A). Further, NOS2/iNOS was identified as a molecular classifier that accurately discriminates between AD and psoriasis using RNA Sequencing (AUC=0.97) and real-time PCR (AUC=1) platforms (FIG. 2).

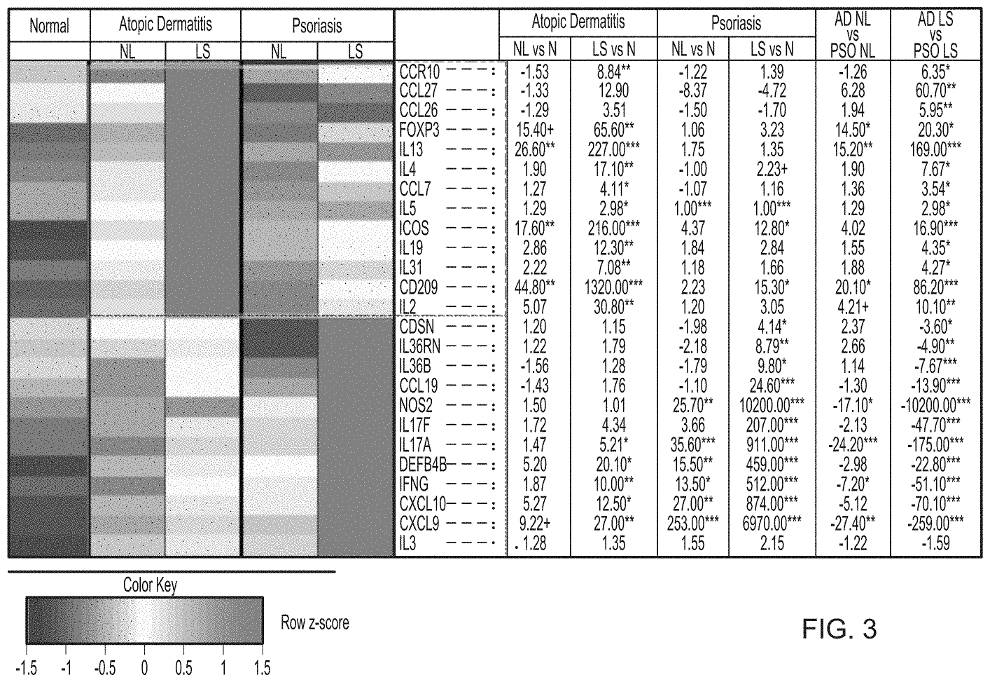

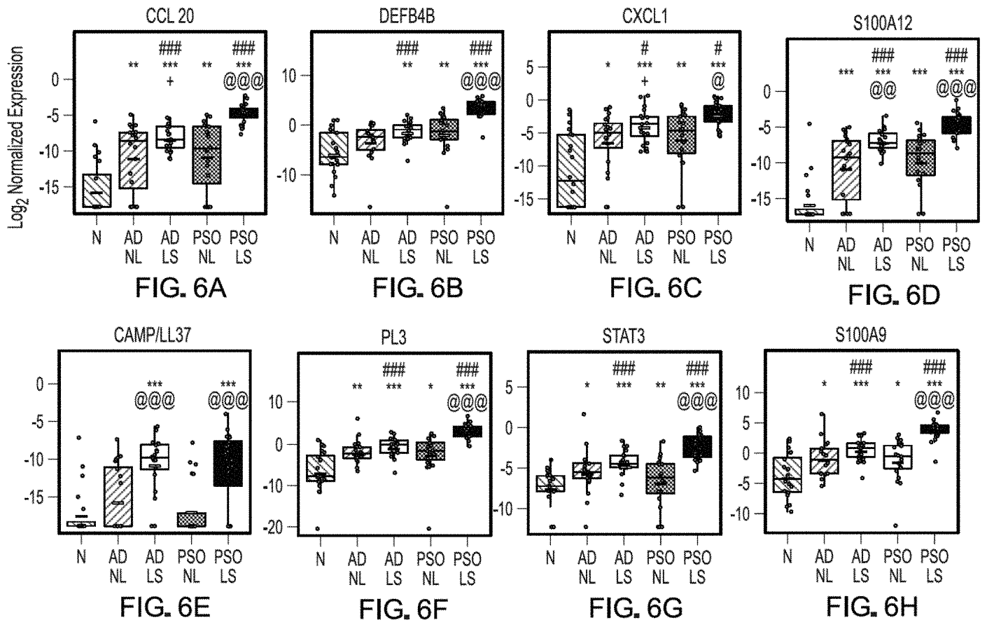

In contrast, Th2 cytokine genes were consistently significantly up-regulated in lesional and/or non-lesional AD versus controls and psoriasis tissues. These included markers that matched RNA-seq data (IL-13, IL-31, CCL17/TARC, CCL22), as well as others that were below detection in RNA-seq, including the key Th2 cytokine IL-4, and mediators of eosinophil growth/recruitment IL-5 and CCL26/eotaxin-3 (P<0.05). IL-13, CCL17/TARC, CCL22, TNFRSF4/OX40, and IL-1RL1/ST2/IL-33R were also significantly higher in non-lesional AD versus both control and non-lesional psoriasis (P<0.05). Th1 products (IFNG, CXCL9, CXCL10, STAT1) were up-regulated in lesional and/or non-lesional psoriasis versus both controls and AD (P<0.05) (FIG. 4). Th17-related genes were mostly significantly increased in lesional and/or non-lesional psoriasis versus both controls and AD tissues, similar to RNA-seq. IL-17A, IL-23p19, IL-36A, CCL20, DEFB4B, CXCL1/2, and PI3 were also overexpressed in non-lesional psoriasis versus controls (P<0.05) (FIGS. 5-6). Like in RNA-seq, the epidermal cytokine IL-19 was the only IL-17-related product higher in AD than psoriasis. The Th17/Th22-related genes (IL-22, most S100As) were elevated in both diseases, although the S100As were significantly higher in psoriasis versus AD lesions (P<0.05) (FIG. 6). The T-reg genes IL-10 and FOXP3 were higher in AD lesions than both controls and psoriasis (P<0.05), while negative regulators (IL-34, IL-37) were decreased in both diseases versus controls. Epidermal hyperplasia markers (KRT16, SERPINB3, and Ki67) were significantly increased in both AD and psoriasis lesions (P<0.05), although KRT16 and SERPINB3 was higher in psoriasis versus AD (P<0.01). The terminal differentiation products (FLG, FLG2, and LOR) were down-regulated in lesional AD versus controls (P<0.05). A summary heatmap of all evaluated markers is presented in FIG. 3, highlighting predominantly Th2 (upper dashed line box surrounding CCR10 through IL2) and Th1/Th17 (lower dashed line box surrounding CDSN through IL3) cluster of genes preferentially enhanced in AD and psoriasis, respectively. NOS2/iNOS was identified as a single gene classifier, able to discriminate lesional AD from lesional psoriasis with 100% accuracy (area under the curve, AUC=1.0) by qPCR, and almost perfectly (AUC=0.97) using the RNA-seq data (FIG. 2).

### CONCLUSION

The results of this study show that NOS2/iNOS is an effective biomarker for psoriasis and it is able to differentiate AD from psoriasis with 100% accuracy in noninvasively obtained tape-stripped skin samples. Further biomarkers of psoriasis include innate immune biomarkers (IL-17C, IL-1B, IL-6, and CXCL8/IL-8) which are significantly upregulated in tape-stripped psoriatic skin samples. Th1 related biomarkers (IFNg, CXCL9) and Th17 related biomarkers (IL-17A, IL-17F, IL-23p19, IL-12/IL-23p40, IL-36A, IL-36G, DEFB4B, CCL20, CXCL1LL37, PI2, STAT3, S100A9, S100A12), are also upregulated in tape-stripped psoriatic skin samples. Lastly, the biomarkers DEFB4B, CXCL9, SERPINB3, CCL20, S100A9, IL17A, PI3, CXCL10, IL36A can help differentiate AD from psoriasis.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating psoriasis, comprising discriminating between atopic dermatitis and psoriasis in a human subject exhibiting an indeterminate lesion, further comprising:
(a) obtaining a surface skin sample from the subject exhibiting a skin lesion by tape-strip applied to a selected skin surface, wherein application of the tape-strip to the selected skin surface transfers the surface skin sample to the tape-strip;
(b) determining the level of NOS2/iNOS biomarker in the surface skin sample;
(c) comparing the level of the NOS2/iNOS biomarker in the surface skin sample to a reference level of NOS2/iNOS biomarker in a control sample;
(d) determining that the subject has psoriasis if the levels of NOS2/iNOS biomarker is higher than the reference level of NOS2/iNOS biomarkers in the control sample;
(e) determining that the subject has atopic dermatitis if the NOS2/iNOS level in the surface skin sample are similar to or lower than the reference levels in the control sample; and
(f) administering a therapeutically effective amount of a psoriasis therapy to the human subject identified as having psoriasis.

2. The method of claim 1, wherein the subject does not have psoriasis if the NOS2/iNOS biomarker level in the surface skin sample is zero or negligible.

3. The method of claim 1, wherein the subject has psoriasis if the NOS2/iNOS biomarker level in the surface skin sample is not zero or negligible.

4. The method of claim 1, wherein more than one tape-strip is obtained from the same skin surface.

5. The method of claim 4, wherein at least 3 tape-strips are obtained from the same skin surface.

6. The method of claim 1, wherein the selected skin surface is non-lesional skin.

7. The method of claim 1, wherein the selected skin surface is lesional skin.

8. The method of claim 1, wherein the levels of the NOS2/iNOS biomarker are measured by determining the mRNA levels of the NOS2/iNOS biomarker.

9. The method of claim 1, wherein the levels of the NOS2/iNOS biomarker are measured by determining the cDNA levels of the NOS2/iNOS biomarker.

10. The method of claim 1, wherein the levels of the NOS2/iNOS biomarker are measured by determining the protein levels of the NOS2/iNOS biomarker.

11. The method of claim 1, wherein the psoriasis therapy is at least one therapy selected from a group consisting of topical treatment, phototherapy, oral medication, and a biological drug.

12. The method of claim 11, wherein the topical treatment is one or more agents selected from a cream, lotion, spray, moisturizer, bath salt, immunomodulator, coal tar, anthralin, corticosteroid, and vitamin D.

13. The method of claim 12, wherein the cream is calcipotriene cream.

14. The method of claim 12, wherein the moisturizer contains one or more of salicylic acid, lactic acid, petroleum jelly, and paraffin.

15. The method of claim 12, wherein the immunomodulator is tacrolimis or pimecrolimus.

16. The method of claim 11, wherein the biological drug is selected from abatacept, adalimumab, certolizumab, brodalumab, dupilumab, etancercept, golimumab, guselkumab, infliximab, ixekizumab, risankizumab, secukinumab, tildrakizumab and ustekinumab.

17. The method of claim 11, wherein the oral medication is selected from methotrexate, acitretin, cyclosporine, and apremilast.

18. A method of treating atopic dermatitis, comprising discriminating between atopic dermatitis and psoriasis in a human subject exhibiting an indeterminate lesion, further comprising:
(a) obtaining a surface skin sample from the subject exhibiting a skin lesion by tape-strip applied to a selected skin surface, wherein application of the tape-strip to the selected skin surface transfers the surface skin sample to the tape-strip;
(b) determining the level of NOS2/iNOS biomarker in the surface skin sample;
(c) comparing the level of the NOS2/iNOS biomarker in the surface skin sample to a reference level of NOS2/iNOS biomarker in a control sample;
(d) determining that the subject has psoriasis if the levels of NOS2/iNOS biomarker is higher than the reference level of NOS2/iNOS biomarkers in the control sample;
(e) determining that the subject has atopic dermatitis if the NOS2/iNOS level in the surface skin sample are similar to or lower than the reference levels in the control sample; and
(f) administering a therapeutically effective amount of an atopic dermatitis therapy to the human subject identified as having atopic dermatitis.

19. The method of claim 1, wherein the levels of the NOS2/iNOS biomarker are measured by determining the mRNA levels of the NOS2/iNOS biomarker.

20. The method of claim 1, wherein the levels of the NOS2/iNOS biomarker are measured by determining the cDNA levels of the NOS2/iNOS biomarker.

21. The method of claim 1, wherein the levels of the NOS2/iNOS biomarker are measured by determining the protein levels of the NOS2/iNOS biomarker.

22. The method of claim 18, wherein the atopic dermatitis therapy is at least one therapy selected from a group consisting of topical treatment, phototherapy, oral medication, and a biological drug.

23. The method of claim 22, wherein the biological drug is selected from dupilumab, dupixent, traloknumab, upadicitinib, abrocotinib, and lebrikizumab.

24. The method of claim 22, wherein the topical treatment is ruxolitinib.
